(19) United States
(12) Patent Application Publication
(10) Pub. No.: US 2021/0353854 A1
(43) Pub. Date: Nov. 18, 2021

(54) DRUG DELIVERY DEVICE

(71) Applicant: Takeda Pharmaceutical Company Limited, Osaka (JP)

(72) Inventors: Andrew Scott Argersinger, Bristol, WI (US); Daniel Edward Roush, Niles, IL (US); Tejas Jayendra Dhyani, Mundelein, IL (US); Adam Blake Hartman, Denton, TX (US); Brent Whitfield Byers, Plano, TX (US); Angela Teresa Muriset, Hoffman Estates, IL (US); Kimberly Duff, Knoxville, TN (US); Dhairya Kiritkumar Mehta, Waltham, MA (US); Sujit K. Basu, Newton, MA (US); Jennifer Craig Cordova, Highland Park, IL (US)

(73) Assignee: Takeda Pharmaceutical Company Limited, Osaka (JP)

(21) Appl. No.: 17/282,245

(22) PCT Filed: Sep. 24, 2019
(86) PCT No.: PCT/US2019/052577

§ 371 Date: Apr. 1, 2021

Related U.S. Application Data
(60) Provisional application No. 62/819,385, filed on Mar. 15, 2019.
(60) Provisional application No. 62/740,496, filed on Oct. 3, 2018.

(51) Int. Cl. A61M 5/142; A61M 5/158

(52) U.S. Cl. CPC A61M 5/14244; A61M 5/158; A61M 2005/14208; A61M 2205/8256; A61M 2205/3553; A61M 2205/8206

(57) ABSTRACT

An infusion pump which may include a control unit and a pump engine may be used to facilitate administration of medicinal fluids to a patient. A control unit may include a motor, a controller, a housing, and a motor coupling, where the motor coupling includes two protruding motor coupling tabs extending in a direction parallel to an output shaft of the motor. A pump engine may include a housing, a rotor disposed in the housing, and a pump coupling, where the pump coupling includes at least two protruding pump coupling tabs extending in a direction parallel to an input shaft of the rotor. The controller may be configured to change an operational mode of the infusion pump, where when the infusion pump is in a positioning mode, the controller activates the motor to move the output shaft to a predetermined angular position.

### CROSS-REFERENCE TO RELATED APPLICATIONS

This application claims the benefit under 35 U.S.C. § 119(e) of U.S. Provisional Application 62/819,385, filed on Mar. 15, 2019, and U.S. Provisional Application 62/740,496, filed on Oct. 3, 2018, each of which is incorporated herein by reference in its entirety.

### FIELD

Disclosed embodiments are related to a drug delivery device. Some embodiments are related to an infusion pump and an infusion pump coupling.

### BACKGROUND

Medicinal fluids are administered to patients through a variety of methods. These conventional methods typically include injection by a syringe, ingestion, or delivery by an infusion pump and needle. In the case of administration by an infusion pump, controlled volumes of medicinal fluids may be delivered to the patient at pre-programmed rates or automated intervals.

Typically, the delivery of medicinal fluids by an infusion pump is performed by an experienced health care provider who is responsible for operating the infusion pump to administer medicinal fluid as well as any maintenance or cleaning the infusion pump might require. A health care provider will also connect any accessories to the infusion pump to facilitate infusion, including needle sets, tubing, and containers of medicinal fluid.

Conventional infusion pumps can be complex to operate and can require delicate cleaning processes performed by a health care provider. Additional complexity can be added during an administration process which may require the connection of specific needle sets, tubing, and containers of medicinal fluid. Accordingly, conventional infusion pumps lack a streamlined procedure for performing an administration process and maintain the cleanliness of an infusion pump. The inventors have recognized the need for an infusion pump that simplifies administration of medicinal fluid to a patient and cleaning of the infusion pump.

### SUMMARY

In some embodiments, systems and methods for administering medicinal fluids to a patient with an infusion pump having a control unit and a pump engine are provided. In some embodiments, a control unit includes a motor, a controller, a housing, and a motor coupling, where the motor coupling includes at least two protruding motor coupling tabs extending in a direction parallel to an output shaft of the motor. In some embodiments, a pump engine for use with a control unit includes a housing, a rotor disposed in the housing, and a pump coupling, where the pump coupling includes at least two protruding pump coupling tabs extending in a direction parallel to an input shaft of the rotor. In some embodiments, a method for operating an infusion pump includes determining a position of an output shaft of a motor, operating the motor to rotate the output shaft, stopping the motor to align the output shaft and/or motor coupling with a predetermined angular position, and coupling a pump engine to a control unit. In some embodiments, an infusion pump includes a motor, a pump engine, and a controller configured to change an operational mode of the infusion pump, where when the infusion pump is in a positioning mode (e.g., a power on mode) the controller activates the motor to move the output shaft and/or motor coupling to a predetermined angular position (e.g., an indexed home position). In some embodiments, an infusion pump includes a motor with a motor coupling and a pump engine with a pump coupling, where the motor coupling and the pump engine have an angular backlash of at least 20 degrees when operatively connected.

In one embodiment, a control unit includes a motor including an output shaft extending in a first direction, a controller configured to change a state of the motor, a housing configured to receive a pump engine, and a motor coupling disposed on the output shaft. The motor coupling includes at least two protruding motor coupling tabs extending in a direction parallel to the first direction, where the at least two motor coupling tabs are configured to contact a pump coupling of the received pump engine to transmit torque from the motor to the pump engine.

In another embodiment, a pump engine for use with a control unit includes a housing and a rotor disposed in the housing and including an input shaft extending in a first direction, where the rotor is configured to pump fluid when rotated about the input shaft. The pump engine also includes a pump coupling disposed on the input shaft, the pump coupling including at least two protruding pump coupling tabs extending in a direction parallel to the first direction. The pump engine is configured to be operatively connected to the control unit, and the at least two pump coupling tabs are configured to contact a motor coupling of the control unit to transmit torque between the rotor and the control unit.

In another embodiment, a method for operating an infusion pump includes supplying power to a controller and a motor including an output shaft and a motor coupling, determining the angular position of the output shaft and/or motor coupling with at least one sensor connected to the controller, operating the motor with the controller to rotate the output shaft and/or motor coupling toward a predetermined angular position, stopping the motor with the controller to stop rotation of the output shaft and/or motor coupling when the output shaft and/or motor coupling is aligned with the predetermined angular position, and coupling a pump engine to a control unit to operatively connect the pump engine to the output shaft via the motor coupling. In one embodiment, the angular position of the output shaft may be determined and the motor coupling aligned with a predetermined angular position. Such an arrangement may ensure slippage does not affect the alignment between the motor coupling and the pump engine when the pump engine is operatively connected to the output shaft. Of course, the angular position of one or both of the output shaft and motor coupling may be determined and controlled, as the present disclosure is not so limited.

In another embodiment, an infusion pump includes a motor including an output shaft with a motor coupling and a pump engine including a rotor with a pump coupling, where the pump coupling and the motor coupling are configured to operatively connect the output shaft to the rotor when the motor coupling receives the pump coupling. The infusion pump also includes a controller configured to change an operational mode of the infusion pump, where in a pumping mode the controller activates the motor to transmit torque through the output shaft, and where in a positioning mode (e.g., a power on mode) the controller activates the motor to move the output shaft to a predetermined angular position.

In another embodiment, an infusion pump includes a motor having an output shaft with a motor coupling and a pump engine including a rotor with a pump coupling. The motor coupling includes a first contact surface and the pump coupling includes a second contact surface. The pump coupling and the motor coupling operatively connect the output shaft to the rotor via contact between the first contact surface and the second contact surface. The pump coupling and the motor coupling have at least 20 degrees of angular backlash between the first contact surface and the second contact surface.

In one embodiment, an infusion set includes an administration set having a first tubing, an inlet, and an outlet. The infusion set also includes a pump engine being pre-connected to the administration set. The infusion set also includes a needle set comprising a first end and a second end, the second end comprising a needle. The first end of the needle set is connected to the outlet of the administration set.

In one embodiment, a medical delivery device for delivering a medicinal substance into a user's body includes a foldable hub having a left wing and a right wing, the hub being attached at one end to a tube, and attached at an opposite end to a needle. The left wing has a first side and a second side, and the right wing has a first side and a second side. The left and right wings have a first position in which the needle is positioned between the left and right wings, and a second position in which the wings are bent away from the needle.

In one embodiment, an infusion pump includes a housing configured to receive a pump engine, a motor disposed in the housing and configured to transmit power to the pump engine, a controller disposed in the housing and configured to change a state of the motor, and a battery disposed in the housing, where the infusion pump is configured to deliver between 5 and 60 mL of a medicinal fluid having a viscosity between 10 and 30 cP at 35° C. on a single battery charge. For example, the infusion pump may be configured to deliver between 5 and 50 mL of CUVITRU on a single battery charge. In other embodiments, an infusion pump of exemplarily embodiments described herein may be configured to deliver between 26.25 mL and 1260 mL of a medicinal fluid having a viscosity between 1 and 4.54 cP at 35° C. on a single battery charge. For example, the infusion pump may be configured to deliver between 26.25 and 1260 mL of HYQVIA components (Hyaluronidase and Immunoglobulin). In some embodiments, an infusion pump may including one or more programmable parameters that change the pumping characteristics of the infusion pump to be well suited for different types of volumes of medicinal fluid.

In one embodiment, a method of operating an infusion pump is provided. The method includes providing an infusion pump having a housing, a motor, and a controller, and removably coupling a pump engine to the motor. The method also includes sending (e.g., from a mobile device) a control command to the controller to cause the infusion pump to perform a medication administration action. The method also includes decoupling the pump engine from the motor.

In one embodiment, a system is provided, where the system includes a drug delivery device having a housing, a battery, and a controller. The system also includes a charging dock that is configured to cooperate with the drug delivery device to provide power to the battery of the drug delivery device. Data is configured to be transferred between the drug delivery device and the charging dock, and the drug delivery device is operable to dispense a medication without being connected to the charging dock. In some embodiments, the system may include a charging accessory (e.g., power adaptor and cable) configured to allow the battery to be charged from a standard AC plug. Such an arrangement may also allow the system to operate under power from the AC plug as opposed to the battery.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. In the drawings:

FIG. 1 depicts a perspective view of an embodiment of an infusion pump;

FIG. 2 depicts a perspective view of the infusion pump of FIG. 1 with a compartment lid removed;

FIG. 3 depicts a front view of the infusion pump of FIG. 1 with a compartment lid removed;

FIG. 4A depicts a top view of the infusion pump of FIG. 1 with a compartment lid removed;

FIG. 4B depicts a top view of the infusion pump of FIG. 1 with a compartment lid and a pump engine removed;

FIG. 5 depicts an exploded view of the infusion pump of FIG. 1;

FIG. 6A depicts a perspective view of an embodiment of a motor coupling;

FIG. 6B depicts a bottom view of the motor coupling of FIG. 6A;

FIG. 7A depicts a perspective view of an embodiment of a motor and a motor coupling;

FIG. 7B depicts a front view of the motor and motor coupling of FIG. 7A;

FIG. 8 depicts a perspective view of an embodiment of a pump engine;

FIG. 9A depicts a front bottom perspective view of the pump engine of FIG. 8;

FIG. 9B depicts a bottom view of the pump engine of FIG. 8;

FIG. 10A depicts a top view of an embodiment of a motor coupling;

FIG. 10B depicts a top view of an embodiment of a pump coupling;

FIG. 11A depicts a schematic showing an embodiment of a motor coupling and pump coupling during a coupling process;

FIG. 11B depicts a schematic showing the motor coupling and pump coupling of FIG. 11A during a pumping process;

FIG. 12 is a block diagram of an embodiment of a method for operating an infusion pump;

FIG. 13 depicts another embodiment of a motor coupling and a pump engine;

FIG. 14 depicts yet another embodiment of a motor coupling and a pump engine;

FIG. 15 depicts an embodiment of an infusion set;

FIG. 16 depicts an embodiment of an administration set;

FIG. 17A depicts an embodiment of a needle set;

FIG. 17B depicts another embodiment of a needle set;

FIG. 18 depicts an embodiment of a needle hub;

FIG. 19 depicts an embodiment of a spike adapter;

FIG. 20 depicts the spike adapter of FIG. 19 being connected with an infusion set;

FIG. 21 depicts a perspective view of another embodiment of an infusion pump;

FIG. 22 depicts an exploded view of the infusion pump of FIG. 21;

FIG. 23A depicts a front perspective view of the infusion pump of FIG. 21 and an embodiment of a holster;

FIG. 23B depicts a rear perspective view of the infusion pump and holster of FIG. 23A;

FIG. 24 depicts another embodiment of an infusion set;

FIG. 25 illustrates the use of the infusion pump of FIG. 21 with a patient and in communication with external devices and various parties according to one aspect;

FIG. 26A depicts another embodiment of an infusion pump that is controlled by a mobile device according to one aspect;

FIG. 26B depicts the infusion pump of FIG. 26A communicating wirelessly with a mobile device;

FIG. 27A depicts a front view of another embodiment of a needle hub in a first position;

FIG. 27B depicts a perspective view of the needle hub of FIG. 27A in a second position;

FIG. 28A depicts a front view of another embodiment of a needle hub in a first position;

FIG. 28B depicts a perspective view of the needle hub of FIG. 28A in a second position;

FIG. 29A depicts a front view of another embodiment of a needle hub in a first position;

FIG. 29B depicts a perspective view of the needle hub of FIG. 29A in a second position;

FIG. 30A depicts a front view of another embodiment of a needle hub in a first position;

FIG. 30B depicts a perspective view of the needle hub of FIG. 30A in a second position;

FIG. 31A depicts a front view of another embodiment of a needle hub in a first position; and

FIG. 31B depicts a perspective view of the needle hub of FIG. 31A in a second position.

### DETAILED DESCRIPTION

During a typical administration process of medicinal fluid using an infusion pump, a nurse or other health care provider performs numerous steps to prepare the infusion pump for operation, operate the pump to deliver the medicinal fluid, and maintain the pump for subsequent administrations. At each step, the health care provider takes care to maintain sterility as fittings, medicinal fluid containers, and other accessories are connected and disconnected from the infusion pump. Generally, the health care provider undertakes regularly scheduled cleanings of an infusion pump which are complex processes which require numerous steps to complete. Accordingly, conventional infusion pumps are difficult to operate and typically require time consuming steps to set up, operate, and clean.

In some cases, due to the type, duration and/or frequency of treatment using some medicinal fluids, self-administration can be a desirable option for convenience and/or cost. Infusion pumps which are already complex and difficult to operate for health care providers are even more challenging to operate and maintain for a patient practicing self-administration. For example, a patient may need to connect new needle sets for each treatment, align and connect tubing to the pump, handle and connect one or more containers of medicinal fluid, program a control unit, and perform other steps for a single administration which may be difficult and time consuming. Additionally, a patient may need to perform complicated cleaning and maintenance processes to prepare their infusion pump for subsequent administrations. Accordingly, reducing the complexity and improving the sterility of medicinal fluid administration by an infusion pump is desirable for both health care providers and self-administering patients.

In view of the above, the inventors have recognized the benefits of a control unit and a removable pump engine that may be used in combination to form an infusion pump that delivers a medicinal fluid in an easy to operate manner. The control unit and pump engine may include an intermediary coupling which allows for easy connection and removal of the pump engine from the control unit.

According to exemplary embodiments described herein, an infusion pump, administration set, and needle set may be used with any number of medicinal or nutritional fluids which are delivered to the body (e.g., subcutaneously). In some embodiments, an infusion pump, administration set, and needle set may be configured to deliver Immune Globulin Infusion 10% (Human), Immune Globulin Subcutaneous (Human) 20% (e.g., CUVITRU), Recombinant Human Hyaluronidase (e.g., HYQVIA), and/or other blood products. Without wishing to be bound by theory, infusion pumps of exemplary embodiments herein may be configured to deliver medicinal fluids having a viscosity between 10 and 30 cP at 35° C. In other embodiments, infusion pumps of exemplary embodiments described herein may be configured to deliver medicinal fluids having a viscosity between 1 and 4.54 cP at 35° C. Of course, an infusion pump, administration set, needle set, and associated accessories may be employed with any desirable medicinal fluid, as the present disclosure is not so limited.

In some embodiments, a control unit includes a housing, a motor including an output shaft, a controller configured to change the state of the motor, and a motor coupling disposed on the output shaft. The motor coupling may include at least two protruding motor coupling tabs extending in a directional parallel to the output shaft. The control unit may be configured to receive a pump engine which includes a pump coupling. The at least two motor coupling tabs may be configured to contact a pump coupling of the pump engine to transmit torque between the motor and the pump engine. Such an arrangement may allow the pump engine to be connected and removed from the control unit without inhibition from the motor coupling.

In some embodiments, a pump engine includes a housing, a rotor disposed in the housing having an input shaft, and a pump coupling disposed on the input shaft. The pump coupling may include at least two protruding pump coupling tabs extending in a direction parallel to the input shaft. The pump engine may be configured to be operatively connected to a control unit when the at least two pump coupling tabs contact a motor coupling of the control unit to transmit torque between the rotor and the control unit. Such an arrangement may allow the pump engine to be connected and removed from the control unit easily and in a repeatable manner.

The inventors have also recognized the benefits of a control unit with a pumping mode and a positioning mode to improve the reliability and simplicity of coupling a removable pump engine to the control unit. In the positioning mode, the control unit may move an output shaft of a motor to a predetermined angular position. When the output shaft is in the predetermined angular position, a pump engine may be received and operatively connected without interference from the motor and motor coupling. Thus, the positioning mode may promote easy repeatable connection of a removable pump engine.

In some embodiments, a method for operating a control unit includes supplying power to a controller and a motor including an output shaft, determining the angular position of the output shaft with at least one sensor connected to the controller, and operating the motor with the controller to rotate the output shaft toward a predetermined angular position. The method may also include stopping the motor with the controller to stop rotation of the output shaft when the output shaft is aligned with the predetermined angular position, and coupling a pump engine to the control unit to operatively connect the pump engine to the output shaft. When the output shaft is in the predetermined angular position, the pump engine may be received and operatively coupled to the control unit without interference. Accordingly, the method may provide a reliable way of connecting a pump engine to a control unit.

In some embodiments, an infusion pump includes a motor having an output shaft and a motor coupling as well as a pump engine having a rotor and a pump coupling. The motor coupling and pump coupling may be configured to operatively connect the motor output shaft to the rotor when the motor coupling is operatively connected to the pump coupling. The control unit may also include a controller configured to change an operational mode of the infusion pump. In a pumping mode the controller may activate the motor to transmit torque through the output shaft. In a positioning mode the controller may activate the motor to move the output shaft to a predetermined angular position. When the output shaft is in the predetermined angular position, the motor coupling and pump coupling may be out of contact so that the control unit does not interfere with the reception or removal of the pump engine. Such an arrangement may simplify the connection or removal of the pump engine.

The inventors have also recognized the benefits of an infusion pump that can be used to deliver multiple medicinal fluids that are combined during administration, either by delivering a mixture of the medicinal fluids or by administering the medicinal fluids concurrently or sequentially in a manner that is easy to operate.

In some embodiments, an infusion pump may be used to administer medicinal fluids which are pooled. For example, an infusion pump may be coupled to a pooling device or multiple containers to deliver volume of medicinal fluid larger than that contained in a single container. An infusion pump may also be used to deliver one or more medicinal fluids that are combined during administration. The infusion pump may be configured to mix multiple medicinal fluids during pumping, or may be used to deliver medicinal fluids concurrently or sequentially. In some embodiments, a pump engine of an infusion pump may be sequentially connected to containers of different medicinal fluids. Alternatively, multiple pump engines may be used with the infusion pump sequentially or concurrently to deliver one or more medicinal fluids.

In some embodiments, a pump engine for use with an infusion pump may be permanently or removably coupled to one or more accessories prior to delivery to a patient so that the pump engine is formed as a part of a delivery set. The pre-coupled accessories may include, but are not limited to, needle sets, tubing, and luer activated connectors. During an administration process, these accessories that may be normally connected individually by hand may be delivered to a patient in combination with the pump engine in a predetermined arrangement in which the components are already connected to one another. Thus, a pre-connected infusion set may obviate a number of steps relating to the connection of accessories that may be individually handled for use with conventional infusion pumps. In some embodiments, an infusion set may be configured for use as a part of a specific administration process. That is, aspects of the infusion set may be determined by characteristics of a particular administration process, including, but not limited to, medicinal fluid type, volume, number of fluids, number of delivery sites, and type of delivery site(s). Accordingly, an infusion set may simplify an administration process by providing a patient or health care provider a pump engine with the appropriate accessories pre-coupled to the pump engine. Of course, the pump engine may be independent of any accessories and may be delivered to a patient separately from a set, as the present disclosure is not so limited.

In some embodiments, a control unit includes a housing, a motor, and a controller. The motor may be disposed in the housing and include an output shaft with a motor coupling disposed thereon. The controller may also be disposed in the housing and may be configured to change an operational state of the motor. The controller may be connected to a user interface located on an exterior of the housing which may be used by an operator to instruct the controller to change an operational mode of an infusion pump. The housing may include a receptacle configured to receive a pump engine which may include a lid, channel, catch, or other alignment and retaining features for removably securing a pump engine to the control unit. The motor and motor coupling may be configured so that the motor coupling is accessible to the pump engine when the pump engine is releasably secured to the control unit. That is, the motor coupling may be configured to operatively connect to a pump coupling of the pump engine when the pump engine is secured to the control unit. The motor coupling may include at least two protruding motor coupling tabs configured to contact at least two pump coupling tabs of the pump coupling to transmit torque from the motor to the pump engine. Such an arrangement may allow for unoccupied circumferential space in the coupling. The unoccupied circumferential space may simplify the connection and removal of the pump engine to or from the control unit as there are fewer engagement surfaces on each of the pump and motor couplings that may interfere during said connection and removal.

In some embodiments, a control unit may include solely non-fluid contacting components. More specifically, the control unit may include a housing, motor, and controller configured to change an operational state of the motor, none of which ever directly contact medicinal fluid. The housing may be configured to receive a replaceable pump engine which may be operatively connected to the motor. The pump engine may retain all components in fluid communication with medicinal fluid and the control unit may simply provide power from the motor to drive the pump engine. Thus, maintenance of the control unit is simplified as cleaning of the control unit may not require the cleaning of any component that contacts medicinal fluid. The pump engine may be configured for easy removal and disposal following each administration process. A new pump engine may be replaced and operatively connected to the control unit prior to the next infusion process, thereby reducing the number of steps for use and maintenance of the control unit.

In some embodiments, a pump engine includes a housing, a rotor with an input shaft, and a pump coupling disposed on the input shaft. The rotor may be disposed in an internal volume defined by the housing and the internal volume may include an inlet and an outlet. The rotor may be configured so that rotation of the rotor by the input shaft moves fluid from the inlet to the outlet. In some embodiments, the rotor may be rotated by the input shaft in a reverse direction to move fluid from the outlet to the inlet. The rotor may be configured as any suitable rotary pumping mechanism, including, but not limited to an internal gear, screw, shuttle block, flexible vane or sliding vane, circumferential piston, flexible impeller, liquid-ring pumps, and centrifugal fan. Of course, the rotor and housing may be configured as any other suitable positive displacement or centrifugal pump, as the present disclosure is not so limited. The pump coupling disposed on the input shaft may be accessible from outside of the housing. For example, the pump coupling may be disposed on an external surface of the housing. The pump coupling may be configured to transmit torque from an external source (e.g., a control unit) to the rotor to move fluid from the inlet to the outlet. The pump coupling may include at least two protruding pump coupling tabs extending in a direction parallel to the input shaft. The at least two pump coupling tabs may be configured to contact at least two motor coupling tabs of a motor coupling of a control unit to transmit torque between the motor coupling and the rotor. As discussed previously, such an arrangement may allow for a significant amount of unoccupied circumferential space on the pump coupling which may be beneficial to avoid interference when manipulating the pump engine to operatively connect to the control unit.

In some embodiments, the pump engine may be disposable and configured for a single use with the control unit. That is, a pump engine may be operatively connected to a control unit for a single administration process and afterwards removed from the control unit and disposed of in an appropriate manner. Accordingly, the pump coupling may be manufactured so that no interference occurs with the control unit when the pump coupling is operatively connected to the control unit. Such an arrangement may simplify and reduce the number of steps for an administration process.

In some embodiments, a coupling (e.g., a motor coupling or pump coupling) includes a disk, a shaft channel, and at least two protruding coupling tabs extending from the disk in a direction away from and parallel to the shaft channel. The disk, shaft channel, and coupling tabs may be arranged symmetrically around a rotational axis of the coupling. The coupling tabs may be configured to contact corresponding coupling tabs of another coupling to transmit torque between the corresponding couplings. The at least two coupling tabs may be constructed as a prismatic shape which extends perpendicularly from the disk. The coupling tabs may have any suitable prismatic shape, including, but not limited to, rectangular, trapezoidal, triangular, and cylindrical. The coupling tabs may be shaped to complement corresponding coupling tabs. That is, the coupling tabs may be shaped to increase the contact area or resilience to torque transmission when the coupling tabs are rotated into the corresponding coupling tabs. For example, if the coupling tabs are rectangular prisms, the corresponding tabs may be trapezoidal or triangular prisms with an inclined face. According to this example, the contact area between the tabs may be altered by the angle of the inclined face which may improve wear characteristics.

In some embodiments, a coupling (e.g., a motor or a shaft coupling) may include a shaft channel and a shaft fastener. The shaft channel may be configured to receive an output shaft or input shaft and transmit torque between the shaft and the coupling. The shaft channel may have a circumferential shape corresponding to a circumferential shape of the shaft to be received. For example, a shaft channel for receiving a keyed shaft may include a corresponding key channel or key. Of course, the shaft channel may have any appropriate shape, including, but not limited to, circular, keyed, D-shaped, hexed, and splined. Depending on the shape of the shaft, the shaft channel may transmit torque between the shaft and the coupling by contact force and/or friction. The shaft fastener may be constructed as any suitable fastener for securing the shaft to the coupling, including, but not limited to, set screws and clamping screws. In some embodiments, a coupling may be formed integrally with an input or output shaft and not include a shaft channel or a shaft fastener.

In some embodiments, corresponding couplings may include an equivalent number of protruding tabs. As discussed previously, the shape of the tabs may be different between corresponding couplings to alter the contact area for wear characteristics. The protruding tabs may be arranged symmetrically about an axis of rotation. For example, a motor coupling and a shaft coupling may each include two tabs arranged on opposite sides of the coupling. The tabs may be configured to rotate in a rotational path about an axis of rotation. To couple corresponding couplings, the tabs of one coupling may be moved into the rotational path of the other coupling. In some embodiments, the tabs may occupy a relatively small angular region of the coupling so that a majority of the circumferential angular space of the coupling is unoccupied. According to this embodiment, the coupling may have a significant amount of backlash. For example, if the couplings each have two tabs, there may be up to approximately 100 degrees of backlash in the coupling depending on the central angle of the sector occupied by each of the tabs. Such an arrangement may allow for simple and reliable operative connection as the unoccupied space provides tolerance for angular misalignment of the couplings.

In some embodiments, a first coupling may include a first contact surface and a second coupling may include a second contact surface. The first contact surface may be any suitable structure which is configured to transmit torque to the second contact surface, such as a protrusion. In some embodiments, a first contact surface may comprise a tab, a tooth, a spline, or any other suitable engaging feature configured to engage with another contact surface. The second contact surface may be part of an engaging feature that is configured to receive the first contact surface. For example, the first coupling may be a male connector and the second coupling may be a female connector. According to this example, the second contact surface may be shaped to male connector. As another example, the first coupling and second coupling may both be genderless. For instance, the first contact surface may abut the second contact surface to transmit torque. Accordingly, in some embodiments, the second contact surface may comprise a tab, a tooth, a spline, or any other suitable engaging feature configured to engage with another contact surface. In some embodiments, a motor coupling may include a first contact surface and a pump coupling may include a second contact surface. In some embodiments, the first contact surface is a protrusion and the second contact surface is an indentation, and vice versa.

In some embodiments, a method for administering a medicinal fluid to a patient includes providing a control unit and a pump engine. As discussed above, the pump engine may be coupled to one or more accessories which are appropriate for a particular administration process, such as needle sets and fluid couplers. The method may include operatively connecting the pump engine to the control unit. In some embodiments, operatively connecting the pump engine may include opening a compartment on the control unit and placing the pump engine in a receptacle disposed in said compartment. The method may also include bringing the pump engine into fluid communication with a container of medicinal fluid via a pooling device, syringe, or other suitable fluidic device. The method may further include supplying power to the control unit and changing the mode of the infusion pump to a pumping mode. In the pumping mode, a motor may be activated to transmit torque to the pump engine, thereby pumping medicinal fluid from the container to a patient.

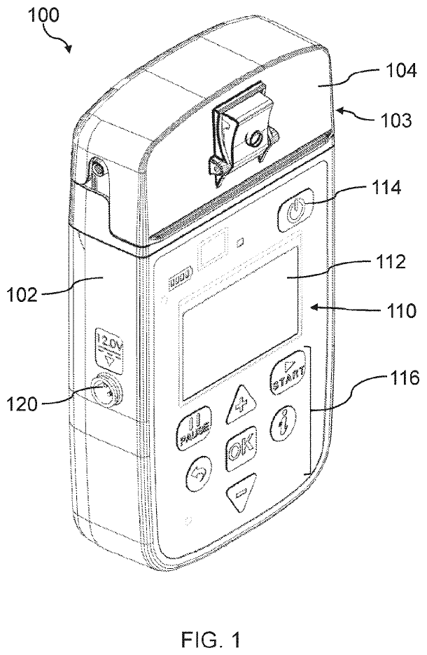

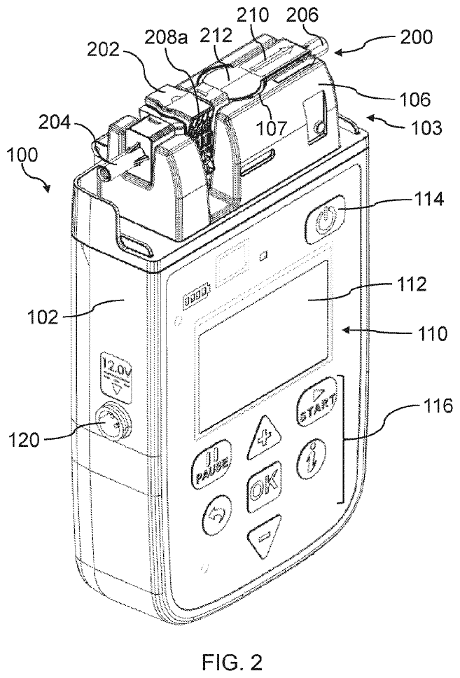

FIG. 1 depicts a perspective view of an embodiment of an infusion pump including a control unit 100 having a housing 102 and a user interface 110 as well as a pump engine (for example, see pump engine 200 in FIG. 2). According to the embodiment of FIG. 1, the housing includes a compartment 103 and contains internal components of the control unit such as a motor and controller. The compartment 103 is covered by an openable compartment lid 104 which is configured to receive a pump engine when opened and secure a pump engine when closed. The user interface 110 includes a display 112, a power switch 114 configured as a power button, and user controls 116. The display 112 is configured to convey information to an operator of the control unit, such as pumping status, instructions for use, alerts, warnings, or any other desirable information. The power button may be used to supply power to a controller and/or motor. As shown in FIG. 1, the control unit includes a power connector 120 for connecting the control unit to a power source configured to supply a source of electrical power. In some embodiments, the control unit may include an internal battery which may be used to selectively supply power to the controller and motor. The user controls 116 may be used to program or select different operational modes of the infusion pump, respond to prompts, or otherwise interact with the controller.

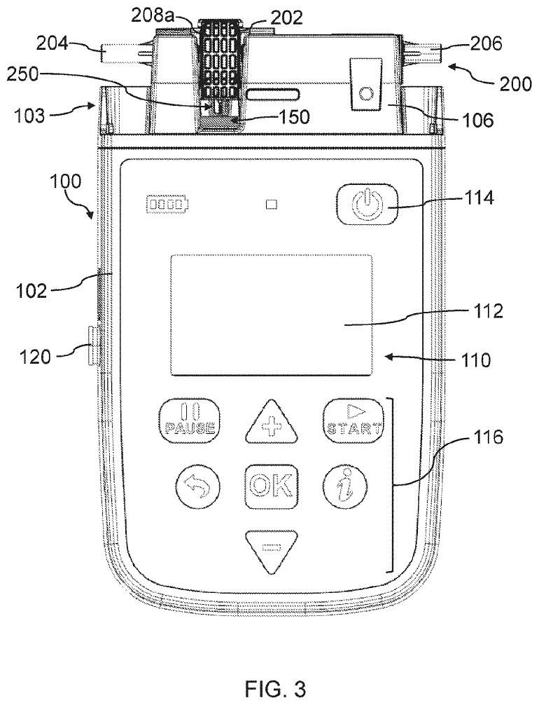

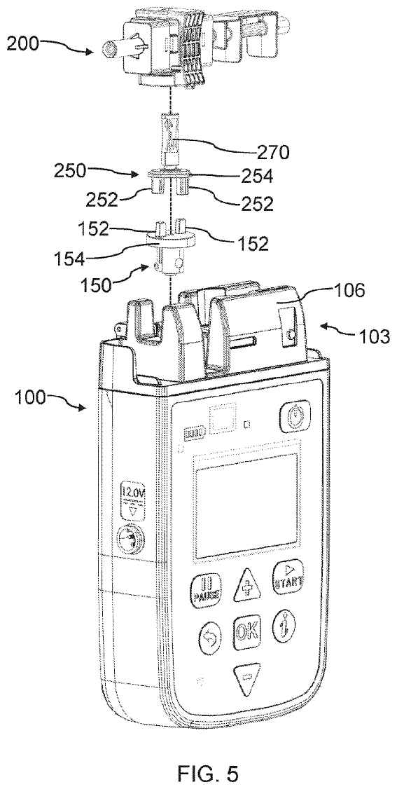

FIG. 2 depicts a perspective view of the infusion pump of FIG. 1 with the compartment lid removed. As shown in FIG. 2, the compartment 103 includes a receptacle 106 configured to receive and secure a pump engine 200. The pump engine includes a housing 202, an inlet 204, and an outlet 206. The pump engine includes a rotor (for example, see FIG. 5) disposed in the housing configured to rotate and move fluid from the inlet to the outlet. As discussed previously, the rotor may be configured as any suitable positive displacement rotor or centrifugal pump rotor. As shown in FIG. 2, the pump engine is non-symmetrical and includes a finger grip 208a and an alignment portion 212 which are configured to interact with the receptacle to align the pump engine for operative connection between the pump engine and the control unit. That is, the receptacle is shaped to receive and orient the pump engine in a correct orientation for alignment between a pump coupling and a motor coupling (for example, see FIG. 3). The receptacle is shaped to complement the shape of the pump engine with the finger grip 208a and the alignment portion 212. Accordingly, the receptacle may prevent the reception of the pump engine unless the pump engine is in a correct orientation, thereby promoting consistent and reliable operative connection between the pump engine and the control unit.

FIG. 3 depicts a front view of the infusion pump of FIG. 1 with the compartment lid removed. As shown in FIG. 3, the pump engine 200 is received in the receptacle 106 so that the pump coupling 250 is brought into operative connection with a motor coupling 150. According to this embodiment, the motor coupling is disposed on an output shaft of a motor located in the housing 102 and projects into the receptacle 106. The pump coupling is disposed on an input shaft of the pump engine which is connected to the rotor of the pump engine. When the pump coupling and motor coupling are brought into operative connection as shown in FIG. 3, torque may be transmitted from the motor in the control unit to the rotor to pump fluid from the inlet 204 to the outlet 206. The torque transmission may be modified depending on the operational mode of the infusion pump which may be set by the controller and user controls 116 of the control unit 100.

A shown in FIG. 3, the pump engine 200 is received in the receptacle 106 which is disposed in the compartment 103. In some embodiments, the pump engine may be secured to the housing outside of any compartment or receptacle of the control unit using one or more catches or latches. In some embodiments, the pump engine may be received by a receptacle but there may be no lid to enclose the pump engine after reception. As shown in FIG. 3, the pump engine is received in a top portion of the control unit. However, the pump engine may be received by any suitable portion of the control unit housing such as the sides or bottom portion.

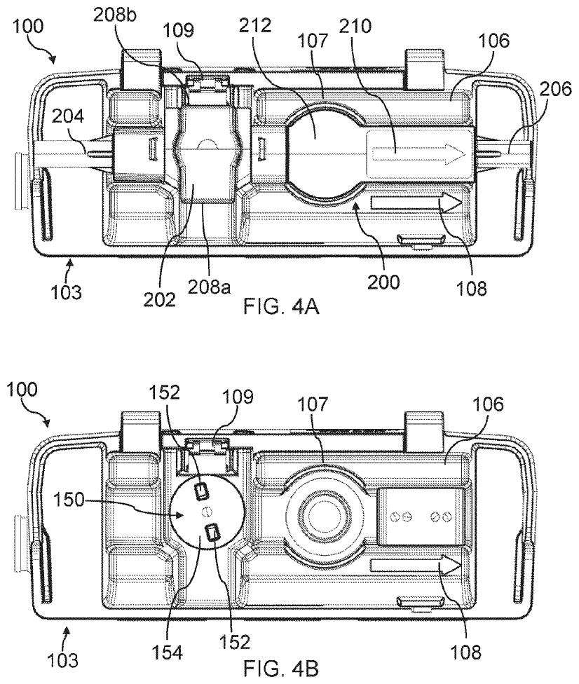

FIG. 4A depicts a top view of the infusion pump of FIG. 1 with the compartment lid removed. As shown in FIG. 4A, the pump engine 200 and receptacle 106 are shaped in a complementary manner such that the pump engine may be received by the pump engine in a single orientation. More specifically, the alignment portion 212 is configured to fit in the alignment receptacle 107 so that the pump engine is properly aligned with the receptacle. According to the embodiment shown in FIG. 4A, the pump engine includes a directional alignment indicator 210 which indicates the correct orientation of the pump engine to an operator. The receptacle similarly includes a directional alignment indicator 108 which may convey to the operator how to orient the pump engine for reception by the receptacle. Once received by the receptacle 106, the pump engine is oriented such that an axis of rotation of a pump coupling is parallel to an axis of rotation of a motor coupling.

According to the embodiment shown in FIG. 4A, the control unit 100 includes a catch 109 configured to releasably secure the pump engine 200 to the control unit. In the depicted embodiment, the catch is configured as a roller catch that engages a second finger grip 208b to apply a positive retention force to the pump engine or otherwise aid a user in installation and removal of the pump engine. In some embodiments, more than one catch may be used to secure the pump engine to the control unit. For example, a second roller catch may be disposed adjacent the finger grip 208a so that positive retention force is applied to both finger grips 208a, 208b. Of course, any suitable number and type of catches may be employed to secure the pump engine to the control unit, including, but not limited to magnetic catches, elbow catches, hooks, slide latches, and ball tension catches.

FIG. 4B depicts a top view of the control unit 100 of FIG. 1 with the compartment lid and the pump engine removed. As shown in FIG. 4B, the motor coupling 150 is disposed at least partially in the compartment 103. The motor coupling includes two motor coupling tabs 152 which extend from a disk 154 in a direction parallel to an output shaft on which the motor coupling is disposed. More particularly, the two motor coupling tabs extend in a direction parallel to an axis of rotation of the motor coupling. The motor coupling tabs are configured to contact similar pump coupling tabs on the pump engine to transmit torque between the pump engine and infusion pump.

FIG. 5 depicts an exploded view of the infusion pump including the control unit 100 of FIG. 1 with the pump engine 200, rotor 270, pump coupling 250, and motor coupling 150 shown removed from the control unit. As shown in FIG. 5, the motor coupling and pump coupling include an equal number of tabs. The pump coupling includes two pump coupling tabs 252 which are shaped similarly to the motor coupling tabs and extend away from the pump engine. The motor coupling is configured to be disposed adjacent to the pump engine when the pump engine is received by the control unit so that the motor coupling tabs and pump coupling tabs are brought into operative connection. That is, the motor coupling and pump coupling are in operative connection when the motor coupling is rotated and the motor coupling tabs and pump coupling tabs contact one another to transmit torque from the motor to the pump engine. In the embodiment of FIG. 5, the motor coupling is at least partly disposed in the receptacle so that when the pump engine is received and aligned by the receptacle the motor and pump couplings are in operative connection. According to the embodiment shown in FIG. 5, the motor coupling and pump coupling may be rotationally positioned relative to one another such that the motor coupling tabs and pump coupling tabs are 90 degrees out of phase. In some embodiments, the motor coupling tabs and pump coupling tabs may be between 40 and 140 degrees out of phase with one another. In this arrangement, the pump engine may be received by the receptacle without interference between the motor coupling tabs and pump coupling tabs.

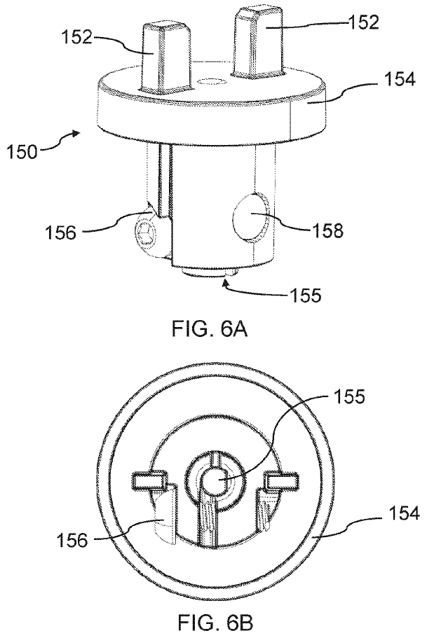

FIGS. 6A-6B depicts a perspective view and a bottom view of an embodiment of a motor coupling 150, respectively. As shown in FIG. 6A, the motor coupling includes two protruding motor coupling tabs 152 which extend from a disk 154. Each of the two tabs forms an approximately rectangular prism which extends perpendicularly from the disk in a direction parallel to that a shaft channel 155. As best shown in FIG. 6B, the shaft channel 155 extends along a longitudinal axis of the motor coupling and is configured to receive a power transmission shaft such as an output shaft from a motor of a control unit. The shaft channel is configured to transmit torque between the power transmission shaft and the motor coupling through friction and/or contact forces. The motor coupling 150 also includes a shaft fastener 156 configured as a shaft clamp. The shaft fastener may be tightened after the shaft is inserted into the shaft channel to clamp the power transmission shaft in the shaft channel. In some embodiments, the motor coupling may include more than two tabs. Of course, any suitable number of tabs may be employed, including, but not limited to a number of tabs between or equal to 1 and 6.

As shown in FIG. 6A, the motor coupling 150 includes a position indicator 158. The position indicator may be used to indicate the angular position of the motor coupling to an associated infusion pump. According to the embodiment in FIGS. 6A-6B, the position indicator may be a magnet and an associated infusion pump may include a Hall Effect sensor configured to detect the position of the magnet. In some embodiments, the position indicator may be a colored patch configured to be detected by an optical sensor disposed in the associated infusion pump. Of course, any suitable position indicator may be employed which may assist an associated infusion pump in determining the position of the motor coupling, as the present disclosure is not so limited.

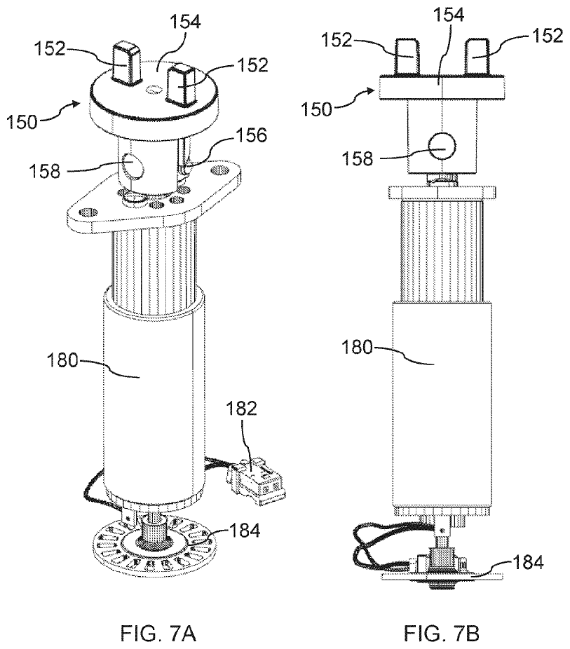

FIG. 7A depicts a perspective view of an embodiment of a motor 180 and a motor coupling 150. As shown in FIG. 7A, the motor coupling is disposed on an output shaft of the motor and is configured to rotate when power is supplied to the motor. The motor includes a power connector 182 which may provide electrical communication between the motor and an associated controller and/or power source. In the embodiment of FIG. 7A, the motor also includes a rotary encoder 184 configured to measure angular displacement of the output shaft of the motor. The rotary encoder may be configured as any suitable rotary encoder which outputs a signal indicative of an angular displacement of the output shaft, including, but not limited to, mechanical encoders, optical encoders, magnetic encoders, capacitive encoders, and incremental encoders. The rotary encoder may be configured to output a signal to an associated controller which the controller may use to determine an angular position of the motor coupling. In some embodiments, the rotary encoder may measure the speed and/or angular displacement of the motor which corresponds to a different output shaft speed and/or angular displacement based on a gear ratio (e.g., gear reduction ratio). According to this embodiment, the associated controller may determine an angular position and/or speed of the output shaft or motor coupling by converting the motor speed using the gear ratio.

FIG. 7B depicts a front view of the motor 180 and motor coupling 150 of FIG. 7A. As discussed previously, the motor coupling includes a position indicator 158 which may be used to determine a particular angular position of the motor coupling. In the embodiment of FIG. 7B, the position indicator is arranged at an angular position 90 degrees offset from the motor coupling tabs 152. Accordingly, when an associated sensor (e.g., a Hall Effect sensor) detects the position indicator, the positioning of the motor coupling tabs may be determined. In some embodiments, multiple position indicators may be used to indicate different angular positions of the motor coupling tabs. The multiple position indicators may be configured so that they may be distinctly detected by a sensor. For example, the position indicator 158 may be a large magnet and another smaller magnet may be disposed in an angular position 0 degrees offset from the motor coupling tabs. According to this example, an associated infusion pump controller may be able to determine one or more absolute positions of the motor coupling tabs which may be beneficial for certain operational modes of an infusion pump.

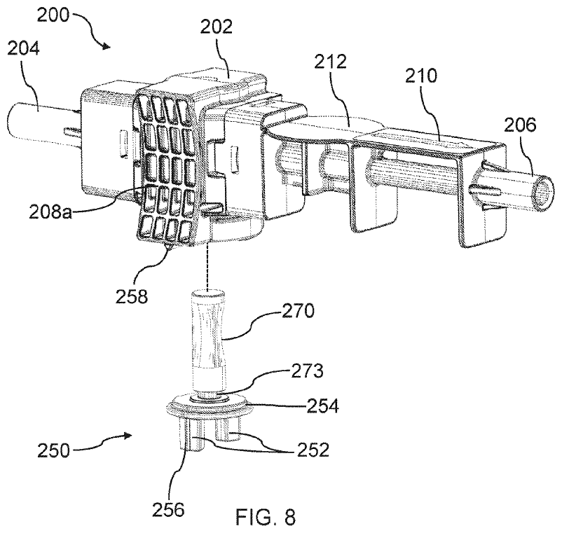

FIG. 8 depicts a perspective view of an embodiment of a pump engine 200. As shown in FIG. 8, the pump engine includes a housing 202, an inlet 204, and outlet 206, and a pump coupling 250. The housing contains a rotor 270 which is configured to pump fluid from the inlet to the outlet when the rotor is rotated. The rotor is coupled to the pump coupling directly or through an input shaft 273 so that rotation of the pump coupling rotates the rotor. According to the embodiment shown in FIG. 8, the rotor is a rotary positive displacement pump with three sealed volumes. Of course, any suitable positive displacement rotor or centrifugal pump rotor may be employed, as the present disclosure is not so limited. The pump coupling includes two pump coupling tabs 252 which extend away from the housing and are configured to contact corresponding motor coupling tabs of an associated infusion pump. As shown in FIG. 8, one of the pump coupling tabs includes a first alignment indicator 256 and the housing includes a second alignment indicator 258. The first alignment indicator and the second alignment indicator may be used to convey an angular offset of the pump coupling relative to a connection position. That is, when the first and second alignment indicators are aligned the pump coupling may be in a connection position. Accordingly, an operator of the pump coupling may be able to determine at a glance whether the pump coupling is in the connection position. In some embodiments, both of the pump coupling tabs may include a first alignment indicator. In this embodiment, the pump coupling may have two connection positions, each offset from each other by 180 degrees. In some embodiments, when the pump coupling tabs are in the connection position the pump engine may be received by an associated infusion pump without inference. More specifically, the pump engine may be received by the associated infusion pump without the pump coupling tabs contacting any components of the control unit. Of course, the pump coupling may have any suitable number of pump coupling tabs, alignment indicators, and connection positions, as the present disclosure is not so limited.

As shown in FIG. 8, the pump engine 200 includes a finger grip 208a disposed on the housing 202. The finger grip is configured to assist an operator in handling and orienting the pump engine. The finger grip forms a concave surface which may be shaped to be easily held with one or more fingers. The finger grip may also be configured to interact with one or more components on an associated infusion pump to releasably secure the pump engine to the control unit. For example, the concave surface of the finger grip may engage a roller catch which applies a retaining force to the finger grip. In some embodiments, a roller catch may provide audible and/or tactile feedback to an operator so that the operator is informed when the pump engine is releasably secured to the control unit. According to the embodiment of FIG. 8, the finger grip is formed in a waffle pattern to reduce weight and material usage in the pump engine. Of course, the finger grip portion may be any suitable surface to facilitate handling of the pump engine by an operator, including, but not limited to, textured surfaces, handles, high-friction surfaces, or other three-dimensional shapes.

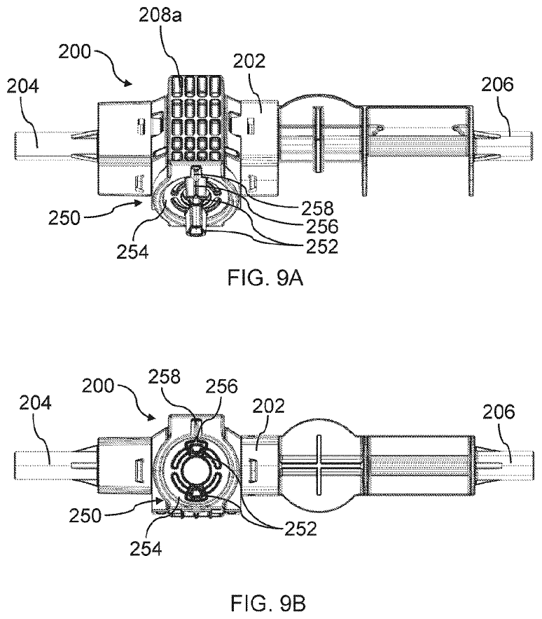

FIGS. 9A and 9B depict a front bottom perspective view and a bottom view of the pump engine of FIG. 8, respectively. As shown in FIGS. 9A-9B, the pump engine includes a pump coupling 250 which is disposed on the housing 202. The pump coupling includes two pump coupling tabs 252 disposed on a disk 254 which extend away from the housing and which are disposed on opposite sides of the pump coupling. According to the embodiment shown in FIGS. 9A-9B, the pump coupling tabs are hollow to reduce the amount of material used in the tabs. However, the pump coupling tabs may be hollow, solid, or any other structure suitable for transmitting torque. As discussed previously, one of the pump coupling tabs includes a first alignment indicator 256 and the housing includes a second alignment indicator 258 which may be used by an operator to determine if the pump coupling is in a connection position.

As best shown in FIG. 9B, the pump coupling tabs 252 are formed as trapezoidal prisms. Each of the trapezoidal prisms includes two inclined faces oriented in a circumferential direction. Such an arrangement may alter the contact patch between the pump coupling tabs and corresponding motor coupling tabs. For example, the inclined faces may be angled so that the face is flush with a face of a motor coupling tab when they are in contact. Depending on the desired contact patch and wear characteristics associated with said contact patch, a suitable predetermined angle may be chosen for each of the inclined faces.

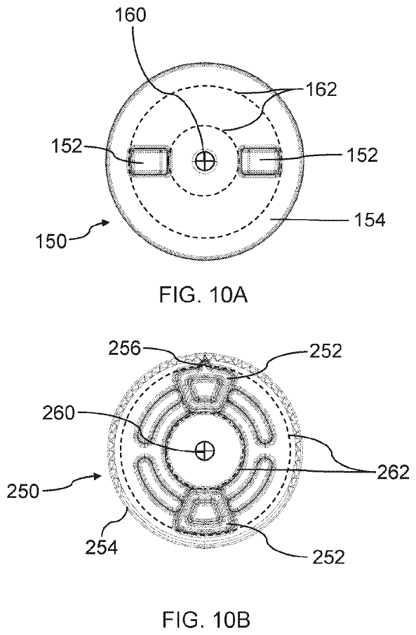

FIG. 10A depicts a top view of an embodiment of a motor coupling 150. The motor coupling includes a disk 154 from which two motor coupling tabs 152 extend. The motor coupling tabs are configured as rectangular prisms and do not include any inclined faces oriented in a circumferential direction. The motor coupling tabs are positioned on opposite sides of an axis of rotation 160 of the motor coupling. Accordingly, as the motor coupling rotates about the axis of rotation the motor coupling tabs rotate in a motor coupling rotational path 162. Any object in the motor coupling rotational path may be contacted by the motor coupling tabs as the motor coupling rotates. The motor coupling tabs may be disposed on opposite sides of an object crossing the motor coupling rotational path, and may transmit torque to the object when the motor coupling is rotated.

FIG. 10B depicts a top view of an embodiment of a pump coupling 250.

Similarly to the motor coupling shown in FIG. 10A, the pump coupling includes a disk 254 from which two pump coupling tabs 252 extend. The pump coupling tabs are formed as trapezoidal prisms with inclined faces oriented in the circumferential direction and are arranged on opposite sides of an axis of rotation 260 of the pump coupling. Accordingly, as the pump coupling rotates about the axis of rotation the pump coupling tabs rotate in a pump coupling rotational path 262. Any object in the pump coupling rotational path may be contacted by the pump coupling tabs as the pump coupling rotates. The pump coupling tabs may be disposed on opposite sides of an object crossing the pump coupling rotational path, and may transmit torque to the object when the pump coupling is rotated. As shown in FIG. 10B, one of the pump coupling tabs includes an alignment indicator which may be used to determine whether the pump coupling is in a connection position.

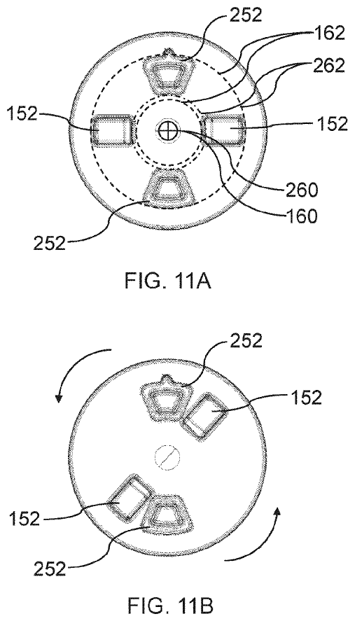

FIG. 11A depicts a schematic showing an embodiment of a motor coupling and pump coupling during a coupling process. A shown in FIG. 11A, the axes of rotation of the motor coupling and pump coupling 160, 260 are aligned and the pump coupling and motor coupling are oriented perpendicularly to one another. Such an arrangement may be appropriate while a control unit receives a pump engine prior to transmitting torque between the control unit and the pump engine. When the pump coupling and motor coupling are perpendicular to each other, the motor coupling tabs 152 and the pump coupling tabs 252 are out of contact with one another. Accordingly, the tabs do not interfere when the couplings are moved in an axial direction relative to each other (for example, see FIG. 5). As shown in FIG. 11A, the axial direction is into the plane of the page, parallel to the axes of rotation 160, 260. Thus, in the position shown in FIG. 11A, the motor coupling and the pump coupling may be moved relative to one another axially along their axes of rotation without causing interference between the motor coupling tabs and pump coupling tabs.

According to the embodiment shown in FIG. 11A, when the axis of rotation 160 of the motor coupling 150 and axis of rotation 260 of the pump coupling 250 are aligned the rotational path of the motor coupling tabs 152 and pump coupling tabs 252 may overlap. That is, the motor coupling rotational path 162 may overlap with the pump coupling rotational path 262 so that the motor coupling tabs may move in the same physical space as the pump coupling tabs when the motor coupling and/or pump coupling are rotated. Accordingly, with the pump coupling and motor coupling and positioned at an appropriate relative axial distance, the motor coupling tabs and pump coupling tabs contact each other to transmit torque between the couplings.

FIG. 11B depicts a schematic showing the motor coupling and pump coupling of FIG. 11A during a pumping process. As shown by the arrows, the motor coupling may be driven by a motor of an associated infusion pump. The motor coupling tabs 152 rotate in a rotational path which is occupied by the pump coupling tabs 252. In the embodiment shown in FIGS. 11A-11B, the motor coupling tabs 152 and the pump coupling tabs 252 have outer diameters that are approximately equal and a majority of their rotational paths overlap. As the motor coupling tabs rotate, they contact the pump coupling tabs simultaneously and apply torque to the pump coupling. In some embodiments, the motor coupling and pump coupling may have a significant amount of backlash to provide a large angular tolerance during relative axial movement of the motor coupling and pump coupling. In the embodiment of FIGS. 11A-11B, the backlash may be approximately equal to 100 degrees. Of course, any suitable amount of backlash may be employed for efficient torque transmission and easy operative connection between the motor coupling and pump coupling, as the present disclosure is not so limited.

In some embodiments, each of the tabs of a motor coupling and pump coupling may occupy a sector of the coupling with a central angle of less than 40 degrees. Such an arrangement may allow for an angular majority of the coupling to be unoccupied. For example, if a coupling has two tabs each occupying a sector of the coupling with a central angle of less than 40 degrees, at least 280 degrees of the circumferential angular space of the coupling is unoccupied by any tabs. If a corresponding coupling also has two tabs each occupying a sector of the coupling with a central angle of less than 40 degrees, there is at least a tolerance of +/−50 degrees from where the corresponding couplings are perpendicular to one another and a position where the tabs would interfere with another when bringing the couplings into operative connection. According to this example and assuming the orientation of each of the corresponding couplings was randomized, it is more likely for a successful connection than a connection which is prevented from interference. Thus, minimizing the number of tabs and the angle of the sector they occupy may have benefits for promoting successful connection of corresponding couplings.

In some embodiments, an appropriate angular backlash between a motor coupling and a pump coupling with two tabs may be greater than or equal to approximately 20 degrees, 30 degrees, 40 degrees, 60 degrees, 75 degrees, 100 degrees, 170 degrees, or any other suitable angular backlash. Correspondingly, an angular backlash between a motor coupling and a pump coupling may be less than or equal to approximately 180 degrees, degrees, 140 degrees, 100 degrees, 60 degrees, 45 degrees, 30 degrees, or any other suitable angular backlash. Combinations of the above noted ranges are contemplated including, for example, angular backlashes between or equal to 40 degrees and 140 degrees, 100 degrees and 170 degrees, 20 degrees and 75 degrees, as well as 20 degrees and 45 degrees. Of course, any suitable angular backlash may be used including angles both greater than and less than those noted above as the present disclosure is not so limited.

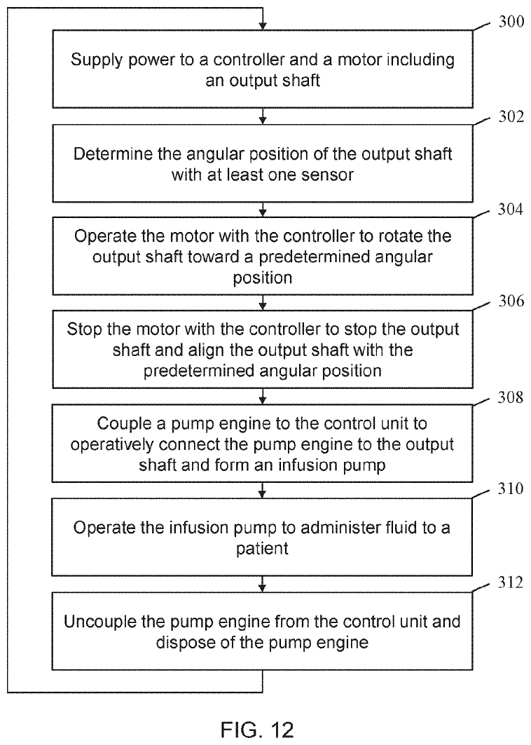

FIG. 12 is a block diagram of an embodiment of a method for operating an infusion pump. In block 300, power may be supplied to a controller and motor of a control unit, where the motor includes an output shaft. The power may be supplied from a power source external to the control unit or an internal battery. In some embodiments, an operator may selectively supply power to the controller and motor using a power switch (e.g., a power button). In block 302, the angular position of the output shaft may be determined using at least one sensor. The position of the output shaft may be determined using information from an angular position sensor, such as a rotary encoder, potentiometer, or other suitable sensor. In some embodiments, the position of the output shaft may be determined by a position indicator disposed on a motor coupling which is connected to the output shaft. The position indicator may be a magnet, optical indicator, or other indicator which may be sensed by a sensor like a Hall Effect sensor or optical sensor. Combinations of sensors may be used for redundancy and increased accuracy to the angular position determination. For example, a rotary encoder may be used in combination with a magnetic position indicator and a Hall Effect sensor. In some embodiments, the angular position of the output shaft may be determined by the controller which may be configured to receive information from the at least one sensor.

In block 304 of the block diagram of FIG. 12, the motor is operated with the controller to rotate the output shaft toward a predetermined angular position. The motor may be operated so that the output shaft rotates more than one full rotation. In block 306, the motor may be stopped with the controller to stop the output shaft so that the output shaft is aligned with a predetermined angular position. The predetermined angular position may be chosen so that when a pump engine is received by the control unit, there is no interference caused by a motor coupling disposed on the output shaft. In block 308, a pump engine is coupled to the control unit to operatively connect the pump engine to the output shaft to form the infusion pump.

In some embodiments, coupling the pump engine may include aligning rotational axes of a motor coupling and a pump coupling and moving the pump engine axially along said axes such that tabs on each coupling are brought into one another's rotational path. When the tabs on each of the motor coupling and pump coupling are bought into one another's rotational path, the tabs of the couplings may contact one another and transmit torque between the motor coupling and the pump coupling as discussed previously. In some embodiments, motor coupling tabs and pump coupling tabs may be angularly out of phase with one another when the output shaft is in the predetermined angular position so that the tabs do not interfere with one another as the pump engine is moved axially along the rotational axes of the couplings. For example, the coupling tabs may be between or approximately equal to 40 and 140 degrees out of phase with the pump coupling tabs when the output shaft is in the predetermined angular position and the at least two pump coupling tabs are moved into the rotational path. As another example, the motor coupling tabs may be approximately 90 degrees out of phase with the two pump coupling tabs when the output shaft is in the predetermined angular position and the pump engine is coupled to the control unit. Of course, the motor coupling tabs and the pump coupling tabs may have any suitable angular offset when the output shaft is in the predetermined angular position, as the present disclosure is not so limited.

In some embodiments, a pump engine including a pump coupling may be manufactured so that the pump coupling is delivered to a patient in a connection position. The connection position of the pump coupling may complement a predetermined angular position of an output shaft of an associated infusion pump. For example, the pump coupling may be in a connection position which is out of phase with a motor coupling disposed on the output shaft when the output shaft is in its predetermined angular position. Accordingly, the motor coupling and pump coupling may not interfere during operative connection if each of the pump coupling and output shaft are in their respective connection position and predetermined angular position. Thus, such an arrangement may be beneficial for easy coupling of the pump engine to a control unit. In some embodiments, the pump engine may include an angular position indicator so that an operator may verify that the pump coupling is in the connection position and take corrective action if the pump coupling is out of the connection position.

In some embodiments, coupling a pump engine to a control unit may include engaging a housing of the pump engine with a catch. For example, the housing of the pump engine may be engaged by a roller catch disposed on the control unit. Of course, any suitable catch or latching mechanism may be employed to removably secure the pump engine to the control unit such as magnetic catches, elbow catches, hooks, slide latches, and ball tension catches, as the present disclosure is not so limited.

In block 310 of the block diagram of FIG. 12, the infusion pump is operated to administer fluid to a patient. Operating the infusion pump may include activating the motor to transmit torque to the coupled pump engine. The pump engine may include a rotor which is spun by the motor, thereby pumping fluid from an inlet of the pump engine to an outlet. The pump engine may be configured to move fluid from one or more contains of medicinal fluid to a patient via a needle set. In some embodiments, the pump engine may be configured to pump medicinal fluid from a pooling device for infusion into a patient's abdominal cavity. In some embodiments, operating the infusion pump may include bringing a motor coupling and a pump coupling into contact. For example, at least two motor coupling tabs may be brought into contact with at least two pump coupling tabs when the motor is activated.

In block 312 of the block diagram of FIG. 12, the pump engine is uncoupled from the control unit and disposed of. Uncoupling the pump engine may include releasing one or more catches or latches, opening a compartment, or otherwise removing any securing elements from the pump engine. After uncoupling, the pump engine may be disposed of in any appropriate manner by an operator. In some embodiments, the method shown in the block diagram of FIG. 12 may be repeated as necessary for subsequent administrations of medicinal fluid.

In some embodiments, the method shown in FIG. 12 may be at least partially performed by a control unit controller which changes one or more operational modes of an infusion pump. The operational modes of the infusion pump may correspond to one or more of the blocks shown in FIG. 12. In some embodiments, in a positioning mode, the infusion pump may perform at least the steps in blocks 304 and 306. More specifically, in a positioning mode, a controller of the control unit may activate a motor to move an output shaft toward a predetermined angular position and stop the motor when the output shaft is in the predetermined angular position. In some embodiments, the controller may also determine the angular position of the output shaft prior to activating the motor. In some embodiments, in a pumping mode, the infusion pump may perform the steps in block 310. More specifically, in the pumping mode, the controller of the control unit may activate the motor to transmit torque to a coupled pump engine. The activation of the motor may bring at least two motor coupling tabs of a motor coupling connected to the output shaft into contact with at least to pump coupling tabs of a pump coupling connected to the pump engine.

In some embodiments, a control unit may have a coupling confirmation mode to ensure proper alignment and coupling of a motor coupling and pump coupling. In the coupling confirmation mode, a controller of the control unit may activate a motor to slowly turn the motor coupling. As the motor coupling is slowly turning, an operator may operatively connect a pump engine with a pump coupling to the control unit. As the operator brings the pump coupling into connection with the motor coupling, the operator may apply pressure to the pump engine until the motor coupling and pump coupling suitably align and come into full operative connection. Upon completion of the operative connection, the controller may stop the motor and the control unit may exit the coupling confirmation mode. In some embodiments, the coupling confirmation mode is activated by a button on a user interface of the control unit and is deactivated by closure of a compartment lid. In some embodiments, in the coupling confirmation mode the motor coupling may rotate at a speed of less than 5 RPM.

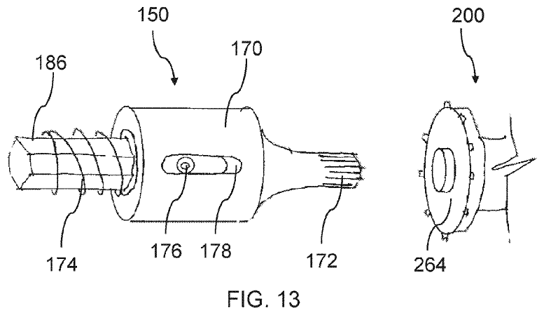

FIG. 13 depicts another embodiment of a motor coupling 150 for operative connection with a pump rotor 264. As shown in FIG. 13, the motor coupling includes a main body 170, a splined connector 172, and a biasing member 174 positioned against a proximal portion of the main body. The main body and biasing member receive a motor output shaft 186 which is slidable relative to the main body. The main body includes a slot 178 and a pin 176 which is slidably disposed in the slot. The pin may be connected to the received output shaft 186 so that the pin is stationary relative to the motor output shaft. The biasing member is configured as a coil spring and may be configured to abut a portion of a motor or a housing of the control unit to apply a distal force to the main body. The biasing member may apply a distal force to the main body so that, in a resting position, the pin 176 rests against a proximal end of the slot 178.

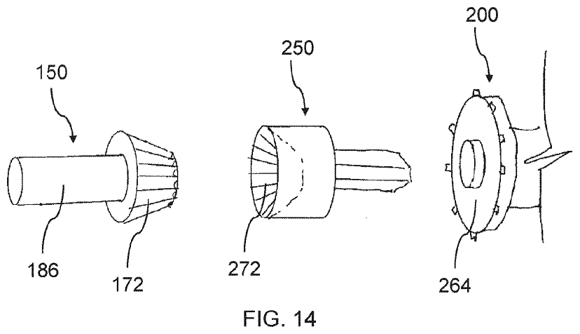

According to the embodiment of FIG. 13, when a pump engine 200 including a pump rotor is brought into engagement with the motor coupling 150, the spline connector 172 may not be suitably aligned with a corresponding pump coupling (for example, see FIG. 14) connected to the rotor 264. Thus, as the pump engine is moved toward the motor coupling, the rotor may displace the main body in a proximal direction such that the pin 176 slides towards a distal end of the slot 178 as the biasing member 174 compresses. As a result, when the pump engine is brought to an appropriate linear position for connection but the spline connector is not angularly aligned with the corresponding pump coupling on the rotor, the biasing member is compressed and applies a distal force on the main body. When the motor is activated and the motor rotates the motor coupling, this distal force causes the splined connector to seat in the corresponding pump coupling connected to the rotor once suitable angular alignment is achieved. Accordingly, the motor coupling of FIG. 13 does not need to be angularly aligned prior to activation of the motor as the biasing member will operatively connect the couplings once a suitable relative alignment is reached.

In some embodiments, the motor coupling of FIG. 13 may use a non-splined connector to operatively connect to the rotor 264 of the pump engine 200. For example, the motor coupling may include a Hirth connector, bevel gear connector, hex connector, or any other suitable connector. In some embodiments, the motor coupling may include at least two protruding tabs instead of splines, gears, or facets. The tabs may extend in a direction parallel to an axis of rotation of the motor coupling and transmit torque to a corresponding coupling according to exemplary embodiments described herein.

FIG. 14 depicts yet another embodiment of a motor coupling 150 and a pump coupling 250. The motor coupling includes a bevel gear connector 172 connected to a motor output shaft 186. The pump coupling includes a corresponding bevel gear cup 272 configured to receive the bevel gear. Without wishing to be bound by theory, the bevel gear arrangement may provide more reliable seating of the motor coupling in the pump coupling. The angled face in combination with the circumference of gears may automatically align the axes of rotation as the motor coupling and pump coupling are brought together axially.

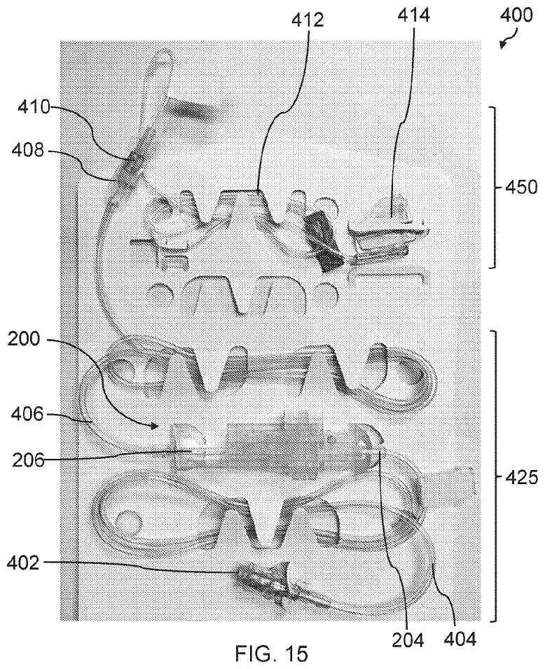

In some embodiments, the pump engine may be configured to couple to an infusion set. The pump engine may, in some embodiments, be pre-connected to an infusion set such that a user need not connect the pump engine to the infusion set. FIG. 15 depicts one illustrative embodiment of an infusion set 400, which includes an administration set 425 and a needle set 450. The administration set 425 includes inlet tubing 404 and outlet tubing 406. As shown in FIG. 15, the inlet tubing 404 is connected to the inlet 204 of the pump engine 200 and the outlet tubing 406 is connected to an outlet 206 of the pump engine. The inlet 204 of the pump engine is fluidly coupled to an inlet connector 402 via the inlet tubing 404. According to the embodiment shown in FIG. 15, the inlet connector is configured as a female luer connector; however, any suitable fluidic connector or valve (e.g., a luer activated valve) may be employed. The outlet 206 of the pump engine is fluidly coupled to an outlet connector 408 via the outlet tubing 406. As shown in FIG. 15, the outlet connector 408 is configured as a male luer connector, and the needle set 450 includes a needle set connector 410 which is configured to mate with the outlet connector. The needle set connector is coupled to needle set tubing 412 which may be a different diameter than at least one of the inlet tubing 404 and outlet tubing 406. The needle set tubing is fluidly coupled to a needle of needle hub 414. Thus, the infusion set is configured to have a continuous fluid pathway from the inlet connector 402 to the needle via the pump engine. According to the embodiment of FIG. 15, the infusion set is pre-coupled so that none of the components of the infusion set are individually handled or coupled prior to a fluid administration. In some embodiments, one or more components of the infusion set may be delivered independent from one another and coupled by a patient or experienced health care provider prior to an administration process.

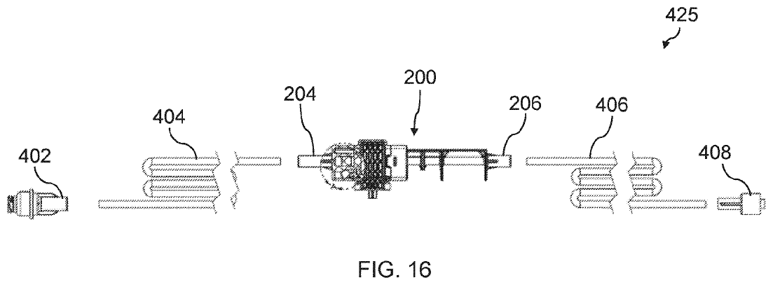

FIG. 16 depicts an exploded view of an administration set 425 and pump engine 200. As shown in FIG. 16, an inlet connector 402 of the administration set is fluidly coupled to an outlet connector 408 via the pump engine 200. The pump engine includes an inlet 204 coupled to the inlet tubing 404 as well as an outlet 206 coupled to the outlet tubing 406. A rotor of the pump engine (for example, see FIG. 8) may be rotated to move fluid from the inlet to the outlet. In some embodiments, the rotor may be rotated to move fluid from the outlet to the inlet. In the embodiment of FIG. 16, the inlet connector and outlet connector are both luer connectors. The inlet connector is configured as a female luer connector and may be configured to connect to a pooling device, syringe, or other medicinal fluid container. The outlet connector is configured as a male luer connector and may be configured to connect to a needle set or other patient interface device. Of course, the inlet and outlet connectors may be any suitable fluidic connectors, including luer activated valves, as the present disclosure is not so limited.

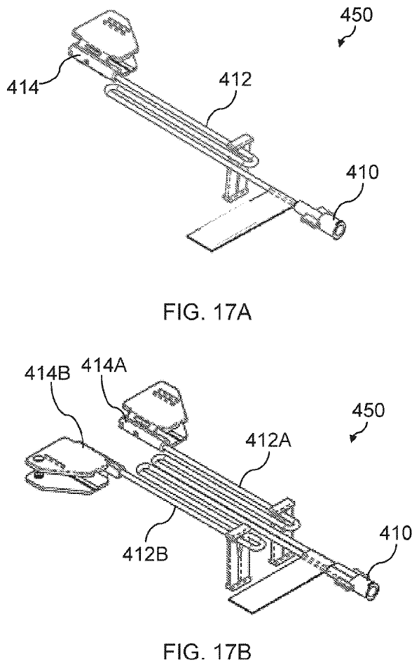

FIG. 17A depicts an embodiment of a needle set 450. As shown in FIG. 17A, the needle set includes a needle connector 410, needle tubing 412, and a needle hub 414 having a needle. According to the embodiment of FIG. 17A, the needle connector is configured as a female luer connector that may connect to a male luer connector (for example, see FIG. 16) of an administration set. Of course, the needle connector may be any suitable fluidic connector, as the present disclosure is not so limited. The needle may be configured as any suitable needle for piercing the skin of a patient.

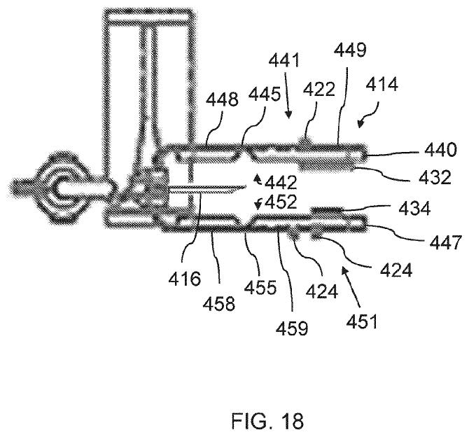

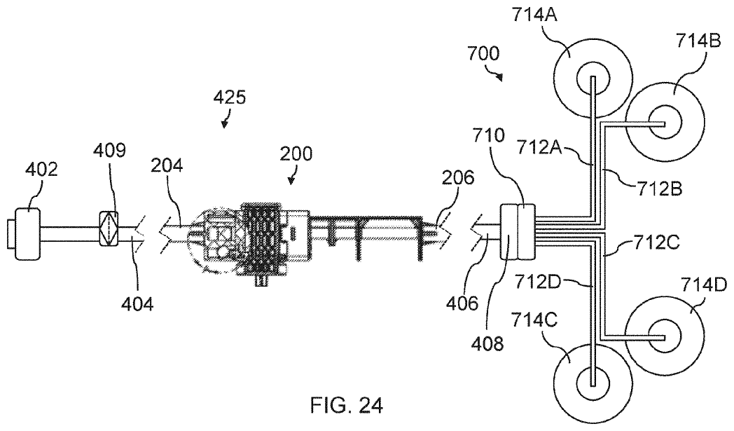

FIG. 17B depicts another embodiment of a needle set 450. As shown in FIG. 17B, the needle set is a bifurcated needle set including two needle tubing sets 412A, 412B and two needle hubs 414A, 414B, each having a needle (for example, see needle 416 in FIG. 18). The needle tubing sets are both coupled to a single needle connector 410 which is configured as a female luer connector that may connect to a male luer connector (for example, see FIG. 16) of an administration set. According to the embodiment of FIG. 17B, the bifurcated needle set may be used to administer medicinal fluid to multiple sites. Such an arrangement may be desirable to reduce the time of administration and/or reduce the localization of the administrated fluid. Of course, while a bifurcated needle set is shown in FIG. 17B, a needle set may include any suitable number of needles to facilitate administration of a medicinal fluid, and may accordingly be bifurcated, trifurcated, quadfurcated (for example, see FIG. 24), quintfurcated, or have any other desirable number of needle hubs.

FIG. 18 depicts an embodiment of a needle hub 414. As shown in FIG. 18, the needle hub includes a needle 416 positioned between a first wing 440 and a second wing 447. The wings 440, 447 are configured such that a user can bend the wings away from the needle 416 to an insertion position to expose the needle and permit the needle to be pierced into the skin of a patient. In the insertion position, the wings are parallel with the needle in a direction opposite a needle extension direction. Accordingly, the wings may be grasped between the fingers to facilitate inserting the exposed needle into the patient's body. In some embodiments, the wings extend in a direction that is perpendicular to the extension direction of the needle in an infusion position. In the infusion position, the wings of the may be secured to a patient's skin with medical adhesives or tape. After infusion, the wings can then be folded back to the position shown in FIG. 18 in which the wings extend in a direction that is parallel to the extension direction of the needle, hereinafter called a safety position. In some embodiments, having the wings in the folded safety position helps to shield the needle from damage and/or protects users from being inadvertently contacted by the needle.

In some embodiments, the needle hub may have one or more features that help to lock the wings in certain positions. In some embodiments, the wings may include locking features that help the wings to remain in an insertion position. In some embodiments, the wings may include locking features that help the wings to remain in a safety position.

In the illustrative embodiment shown in FIG. 18, the wings 440, 447 are configured such that they are biased toward the safety position in which the wings are parallel to one another and to the needle 416. In other words, with the wings at rest, e.g., not locked in place and without an external force being exerted on the wings, they naturally move to the safety position. The wings can be bent to move them out of the way during an infusion procedure to expose the needle 416 (e.g., to the insertion position or infusion position). Each of the wings has a first side and a second side, where the first sides face outwardly when the wings are in the safety position and the second sides face inwardly toward one another and toward the needle when the wings are in the safety position. In some embodiments, the first sides of the wings include one or more protrusions that interact with one another to hold the wings in the insertion position. As shown in FIG. 18, the first side 441 of the first wing 440 includes a single protrusion 422, and the first side 451 of the second wing 447 includes two protrusions 424 that define a gap that is sized to receive and hold the protrusion 422 of the first wing. It should be understood that the protrusion arrangement may be reversed such that the two protrusions are on the first wing and the second protrusions are on the second wing. In other embodiments, other shapes may be used, such as an indentation that receives a protrusion, or a protruding annular rib that receives a smaller protruding annular rib, or other snap fit arrangements.

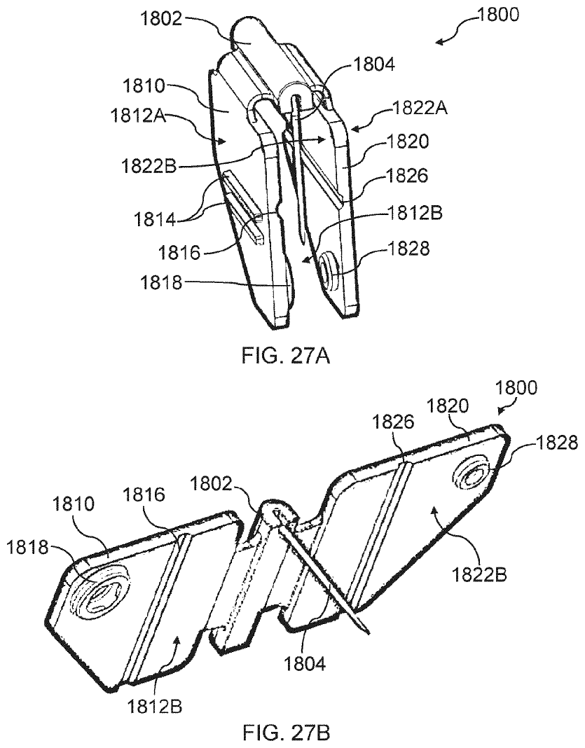

In some embodiments, the wings may include a locking arrangement that keeps the wings further closed over the needle in a closer position than the safety position, hereinafter called a closed position. In some embodiments, in the closed position, the distance between the distal ends of the wings may be smaller than the distance between the distal ends of the wings in the safety position. The second sides of the wings may include ribs, protrusions/indentations, or other snap-fit arrangements that serve to connect the second sides of the wings to one another in the closed position. The wings may be made of flexible material to permit the wings to flex toward one another, and/or the wings may have one or more areas of decreased thickness to permit flex. Exemplary embodiments of needle hubs including various locking features configured to maintain the wings of a needle hub locked in a closed position are described further with reference to FIGS. 27A-31B.

As shown in the illustrative embodiment of FIG. 18, the second side 442 of the first wing 440 includes an annular rib 432 that receives an annular rib 434 on the second side 452 of the second wing 447. The diameter of the annular rib 434 is smaller than that of the annular rib 432 such that the smaller annular rib 434 can be received and snap fit within the larger annular rib 432. Each of the wings 440, 447 includes a flex area 445, 455 that are areas of decreased thickness and help the wings to flex. For the first wing 440, the flex area 445 joins a first portion 448 of the wing to a second portion 449 of the wing. For the second wing 447, the flex area 455 joins a first portion 458 of the wing to a second portion 459 of the wing.

The infusion set may receive medicinal fluid from different types source containers, such as a vial, a syringe, a pooling bag, or a pooling device. One example of a pooling device is described in application Ser. No. 15/186,061, published as U.S. Patent Application Publication No. 2017/0020784, entitled “POOLING DEVICE FOR SINGLE OR MULTIPLE MEDICAL CONTAINERS,” hereby incorporated by reference in its entirety. The infusion set may physically be coupled with a source container such that the infusion set is in fluid communication with the source container. In some embodiments, one or more adapters may be used to permit the infusion set to compatibly couple with different types of source containers. For example, the infusion set inlet may have a luer connector that can connect with source containers having a luer connector, such as a syringe or a pooling device. The inventors have recognized, however, that a user may want to use the infusion set with other arrangements that require the infusion set to pierce into a source container, e.g. directly into a vial or into a pooling bag.

A spike adapter may be connected to the infusion set inlet to permit the infusion set to be used in a spiked configuration. The spike adapter may have a spike on one end and a connector on the other end, the connector being compatible with the infusion set inlet. As an illustrative embodiment, if the infusion set inlet is a female luer connector, the spike adapter connector can be a male luer connector for compatibility with the infusion set.

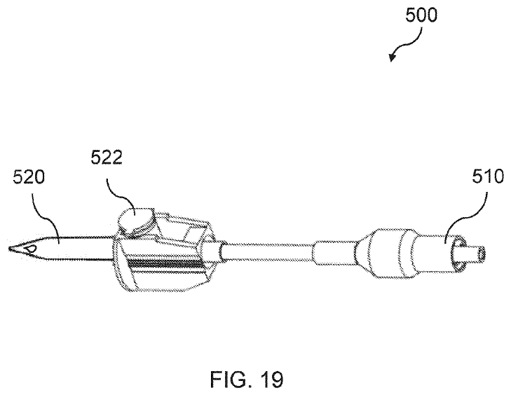

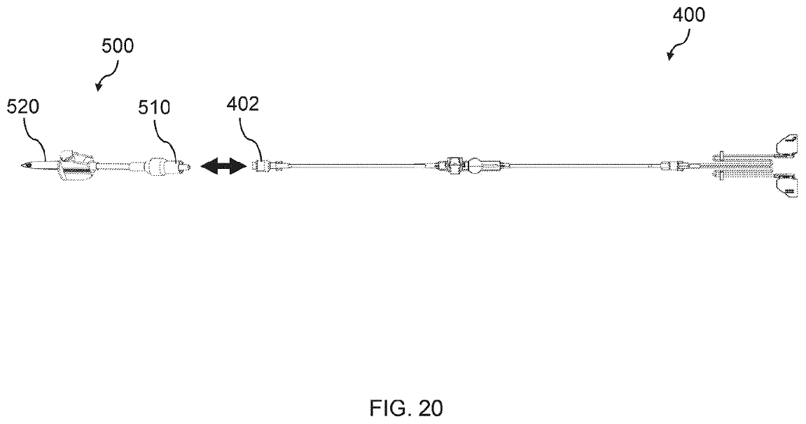

One illustrative embodiment of a spike adapter is shown in FIG. 19. The spike adapter 500 may have a luer connector 510 on one end and a spike 520 on the other, opposing end. The spike may include a vent 522. FIG. 20 shows how the spike adapter 500 connects to an infusion set 400. The inlet end 402 end of the infusion set 400, which is a female luer connector, mates with the male luer connector 510 of the spike adapter 500. After connecting the infusion set 400 with the spike adapter 500, the spike 520 of the spike adapter 500 is in fluid communication with the infusion set 400.

In some embodiments, the infusion pump is portable and can be worn by the patient without needing to connect the pump to an electrical outlet for power during use. For example, the infusion pump may include an internal battery which supplies power sufficient to perform a fluid administration process. The pump may have a small form factor which is sized and shaped appropriately to be coupled to clothing or otherwise worn by a patient during a fluid administration process. Additionally, the pump may have a suitably low weight so that a patient wearing the pump may be mobile during operation of the pump. In some embodiments, the infusion pump may cooperate with a holster which helps a user to wear the infusion pump. The holster may include one or more clips configured to reliably secure the infusion pump to the clothing of a patient (e.g., a belt).

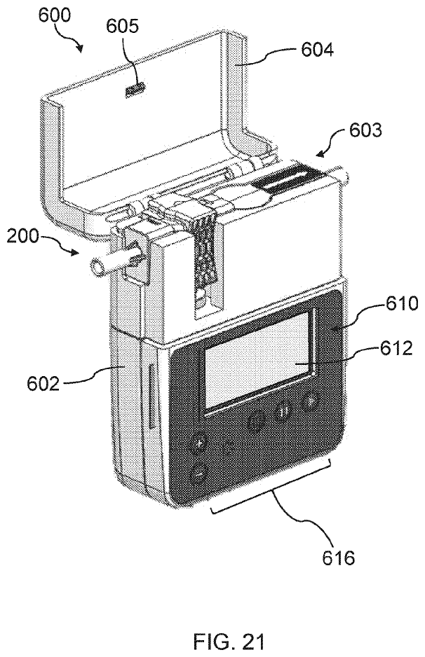

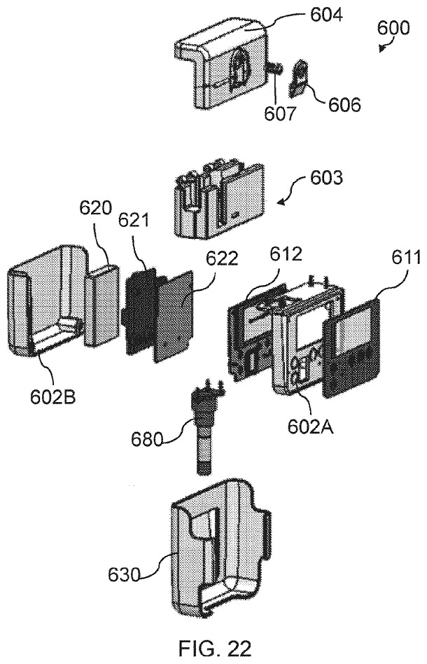

FIG. 21 depicts a perspective view of an embodiment of a small form factor infusion pump 600. According to the embodiment shown in FIG. 21, the infusion pump 600 is similar to the embodiment of FIGS. 1-5 in that the infusion pump is configured to receive a separate pump engine 200. The infusion pump also includes a housing 602, a compartment 603, a lid 604, and a user interface 610. User interface 610 includes a display 612 and a plurality of user controls 616 (e.g., buttons) which may be used to monitor and operate the infusion pump. In the embedment of FIG. 21, the display 612 is configured as an electronic ink display which reduces power consumption of the user interface so that additional battery is reserved for powering the pump engine. The lid 604 includes a latch 605 which may be used to securely retain the lid in a closed position to securely house the pump engine 200 in the compartment 603. In the embodiment of FIG. 21, the small size of the housing 602 may permit the infusion pump to be worn and/or carried by a patient. Additionally, as will be discussed further with reference to FIG. 22, the infusion pump of the present embodiment may be configured to be battery powered for the duration of a medicinal fluid administration session.

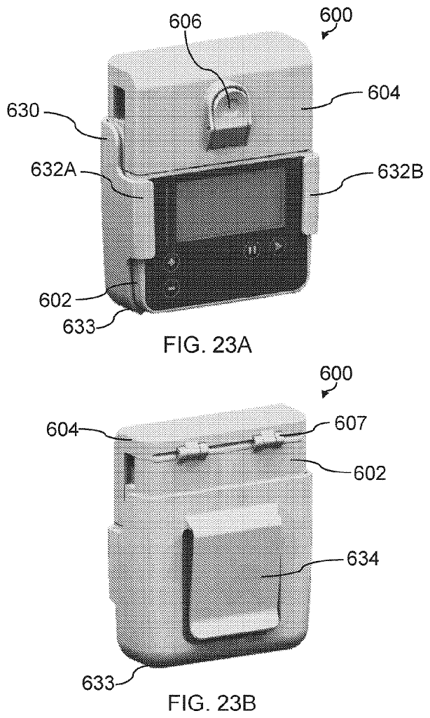

FIG. 22 depicts an exploded view of the infusion pump 600 of FIG. 21. As shown in FIG. 22, the infusion pump housing includes a first housing portion 602A and a second housing portion 602B which contain a motor 680, the display 612, a controller 622, a battery holder 621, and a battery 620. When assembled, a user interface panel 611 is fit over the first housing portion 602A and the compartment 603 is fit over both the first housing portion and second housing portion. According to the embodiment of FIG. 22, the battery may be a high density battery with high amperage throughput to power the motor to pump viscous fluids, such as a lithium polymer battery or other lithium based battery. Of course, the battery may be any suitable battery having an appropriate energy density and power throughput, as the present disclosure is not so limited. The battery may be rechargeable via any suitable external power source, such as an AC-DC power adapter (e.g., USB power adapter) or external battery. The motor 680 may be any suitable geared or direct drive motor with appropriate torque to provide power to the pump engine (see FIG. 21). As shown in FIG. 22, the lid 604 includes a push button 606 which is loaded with a spring 607. The push button may be used to release the latch (see FIG. 21) and is biased by the spring to an engaged state such that the lid stays closed when the compartment 603 is covered. Of course, while a push button and spring are shown in FIG. 22, any suitable latch interface may be employed, as the present disclosure is not so limited. As shown in FIG. 22, the infusion pump may include a holster 630 which may be employed to wear the infusion pump on clothing as will be discussed further with reference to FIGS. 23A-23B.

FIGS. 23A-23B depict a front side and rear side perspective view, respectively, of the infusion pump housing 602 of FIG. 21 disposed in the holster 630. As shown in FIG. 23A, the holster 630 includes a first arm 632A and a second arm 632B which are configured to slidingly receive the housing 602. The holster also includes a base 633 which is configured to stop the sliding motion of the housing into the first arm and second arm. That is, in the state shown in FIG. 23A, the infusion pump housing 602 is fully inserted into the holster such that the first and second arm inhibit transverse motion of the infusion pump housing and the base inhibits motion of the infusion pump housing down relative to the page. Accordingly, in the state shown in FIG. 23A, gravity may be used to retain the infusion pump in the holster and the infusion pump housing may be moved up relative to the page to remove the housing from the holster. In some embodiments the holster may include one or more catches which are configured to releasably retain the infusion pump housing in the fully inserted position.

As best shown in FIG. 23B, the holster 630 includes a clip 634 configured to permit the holster to be attached to clothing. In the present embodiment, the clip 634 is configured as a belt clip which may be releasably attached to a belt or waistband. Accordingly, when the infusion pump housing is disposed in the holster 630, the infusion pump may be worn on clothing during a fluid administration process. As the infusion pump includes a battery (see FIG. 22), the infusion pump may be operated to administer a fluid completely free of any external tethers. In some embodiments, the infusion pump may cooperate with a wearable pooling device or container of medicinal fluid, so that an entire fluid administration system is wearable. Of course, while a belt clip is shown in FIG. 23B, in other embodiments, the holster may include any suitable clips, straps, buckles, snaps, or other arrangements for securing the holster to clothing, as the present disclosure is not so limited. Additionally, in some embodiments, the housing 602 of the infusion pump may include any suitable clips, straps, buckles, snaps, or other arrangements to secure the housing to clothing without a holster, as the present disclosure is not so limited.

According to the embodiment shown in FIGS. 21-23B, the infusion pump may be used by a patient to complete an entire administration process while the infusion pump is worn by the user. That is, the battery (see FIG. 22) may have a sufficient capacity to deliver a prescribed amount of medicinal fluid without an external power source or need for recharging. In some embodiments, the infusion pump may deliver between 5 and 60 mL of a medicinal fluid having a viscosity between 10 and 30 cP at 35° C. on a single battery charge. In particular, an infusion pump may deliver volumes of a medicinal fluid greater than or equal to 5 mL, 10 mL, 20 mL, 40 mL, and/or any other appropriate volume on a single battery charge. Additionally, the infusion pump may deliver fluids of the above-noted volumes with viscosities greater than 1 cP, 2.5 cP, 5 cP, 10 cP, 15 cP, 20 cP, 25 cP, 30 cP, 35 cP, and/or any other appropriate viscosity on a single charge. Correspondingly, the infusion pump may deliver fluids of the above-noted volumes with viscosities less than 50 cP, 45 cP, 40 cP, 30 cP, 20 cP, 15 cP, 10 cP, and/or any other appropriate viscosity on a single charge. In other embodiments, the infusion pump may deliver between 26.25 mL and 1260 mL of a medicinal fluid having a viscosity between 1 and 4.54 cP at 35° C. on a single battery charge. In particular, an infusion pump may deliver volumes of a medicinal fluid greater than or equal to 25 mL, 75 mL, 150 mL, 300 mL, 500 mL, 750 mL, 1000 mL, 1250 mL, and/or any other appropriate volume. Correspondingly, the infusion pump may delivery volume of a medicinal fluid less than or equal to 1500 mL, 1200 mL, 900 mL, 600 mL, 300, mL, 250 mL, 150 mL, 75 mL, and/or any other appropriate volume. Of course, the infusion pump may deliver a volume of medicinal fluid greater than or less than the volumes noted above with any appropriate viscosity, as the present disclosure is not so limited.

According to the embodiment shown in FIGS. 21-23B, the infusion pump may have a suitably small size for extended wearing by a patient without sacrificing pumping functionality. Accordingly, in some embodiments, a total volume occupied by the infusion pump may be less than 300 cm³, 275 cm³, 250 cm³, 225 cm³, 200 cm³, and/or any other appropriate volume. In some embodiments, the total volume of the infusion pump may be between 200 and 300 cm³. Such an arrangement may allow the infusion pump to be easily worn with limited inference with mobility. Of course, sizes greater than or less than those noted above may be employed, as the present disclosure is not so limited.

FIG. 24 depicts another embodiment of an infusion set including an administration set 425 and needle set 700. The administration set is similar to that of FIG. 16, and includes an inlet connector 402 coupled to inlet tubing 404. The inlet tubing is coupled to an inlet 204 of a pump engine 200. An outlet 206 of the pump engine is coupled to outlet tubing 406 which terminates in an outlet connector 408. The needle set 700 is a quadfurcated needle set which includes a needle connector 710 coupled to the outlet connector 408. Accordingly, a fluid channel is formed by the infusion set from the inlet connector 402 to the needle set via needle connector 710. The needle connector quadfurcates the fluid channel into a first needle tubing set 712A terminating in first needle hub 714A, a second needle tubing set 712B terminating in second needle hub 714B, a third needle tubing set 712C terminating in third needle hub 714C, and a fourth needle tubing set 712D terminating in fourth needle hub 714D. Accordingly, the needle set spreads the fluid flow across four separate infusion sites. Such an arrangement may be beneficial to avoid generation of back pressure in the fluid channel and/or increase patient comfort during a fluid administration process. Of course, any suitable number of needles and needle tubing sets may be employed, as the present disclosure is not so limited.

In some cases, it may be desirable to disconnect a first fluid supply and connect a second fluid supply to complete an infusion process while using the same infusion set and/or needle set to deliver medicinal fluid to the patient. For example, the volume of fluid deliverable from a single fluid supply may be insufficient for some dosages. Accordingly, in such cases, it may be desirable to connect another fluid supply safely if an additional volume of fluid is prescribed. Accordingly, an infusion set, needle set, or medicinal fluid tubing according to exemplary embodiments described herein may include a breather valve (i.e., degassing valve, air release valve, etc.) configured to prevent air bubbles which may form during the transition between fluid supplies from being administered to the patient.

In some embodiments, an infusion set includes tubing having a breather valve and a needle set having at least one needle for delivering medicinal fluid to a patient. The infusion set may include an inlet connector which allows the infusion set to be coupled to a fluid supply either directly (e.g., to a fluidic outlet connector of the pooling device) or indirectly (e.g., via an infusion pump, regulator, or other device). Once connected to the fluid supply, the degassing valve may allow air to flow out of the tubing to allow medicinal fluid from the fluid supply to replace said air. Accordingly, air will be removed from the tubing of the infusion set before administration to the patient. Any additional air bubbles which form may also be removed from the tubing by the breather valve.

According to the embodiment of FIG. 24, the administration set 425 includes a breather valve (i.e., degassing valve, air release valve, etc.) 409 configured to allow the administration set and needle set 700 to be sequentially coupled and decoupled with one or more fluid supplies (e.g., pooling devices, fluid containers, etc.). According to the embodiment of FIG. 24, the breather valve is positioned in-line with the inlet tubing 404. The breather valve is configured to vent air pockets that may be disposed in the inlet tubing. For example, when the infusion set is first used with a fluid supply, air inside the infusion set may be vented to prime the pump engine 200 and/or allow fluid to flow from the fluid supply. Additionally, the breather valve may vent additional air bubbles that may form during fluid delivery to inhibit passage of air in the fluid stream. When the infusion set is disconnected from a first fluid supply (e.g., first pooling device), the infusion set may refill at least partially with air. Accordingly, when the infusion set is connected to a second fluid supply (e.g., second pooling device), the breather valve may once again vent air disposed in the infusion set to allow a continuous fluid stream. Accordingly, the breather valve may permit swapping of fluid supplies without requiring a patient to switch infusion sets.

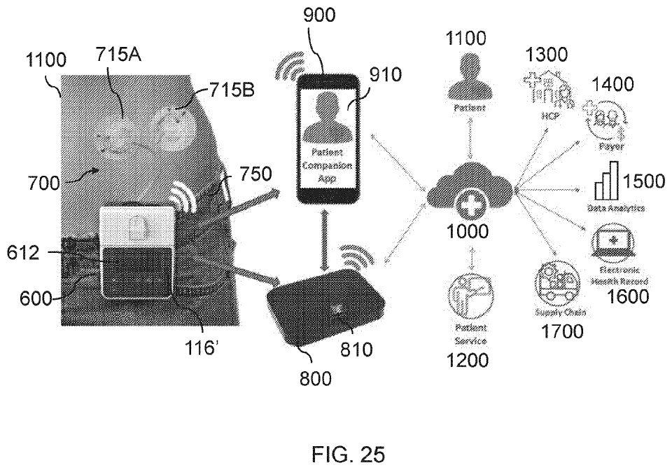

FIG. 25 illustrates the use of the infusion pump of FIG. 21 with a patient 1100. The infusion pump 600 may be coupled to an article of clothing 750 worn by the patient 1100 to allow the patient to remain mobile during medicament administration by the infusion pump. In the illustrative embodiment of FIG. 25, the infusion pump is clipped to the waistband of the patient's pants. In this particular illustrative embodiment of FIG. 25, the needle set 700 is a bifurcated needle set with a first needle hub 715A and a second needle hub 715B to permit two administration sites. However, as discussed previously, other needle sets may be used with the infusion pump, having any suitable number of needle hubs.

According to one aspect, an infusion pump may be in communication with one or more external devices. Information relayed through such communication may be shared with one or more parties, which may use the information in different ways.

Examples of external devices include, but are not limited to: a mobile device such as a smartphone, laptop, tablet, a charging dock, and a remote server.

According to one aspect, a drug delivery device may communicate with an external device that may also serve to charge or otherwise provide power to the drug delivery device. In some embodiments, the drug delivery device may physically connect with the external device with a connector which may transfer data and/or power between the drug delivery device and the external device. Communication may be one-way in either direction, or two-way. Alternatively or in addition, communication and/or power transfer between the drug delivery device and the external device may occur wirelessly.

As used herein, a drug delivery device may include, but is not limited to, an infusion pump, a pen injector, an autoinjector, an inhaler, a nasal spray, and an eye drop dispenser.

As discussed above, some embodiments of the infusion pump include a portable device that may be wearable by a patient. The infusion pump may have a power source that is fully encapsulated within the infusion pump housing in the form of a rechargeable battery. In some embodiments, a charging dock may be provided for ease of recharging the infusion pump. In some embodiments, connecting the infusion pump with the charging dock to charge the infusion pump may also initiate a data transfer process, in which data may be transferred from the infusion pump to the dock, and/or vice versa. The inventors have appreciated that, because a user may need to recharge the infusion pump periodically to be able to operate the infusion pump, integrating data and power transfer within a single charging dock may be a convenient way to ensure periodic data transfers that do not necessarily require the infusion pump to have a wireless communication capability.

It should be appreciated, however, that in some embodiments, the infusion pump may have built-in wireless communication capabilities. The infusion pump may communicate wirelessly to a charging dock. In some embodiments, the infusion pump may use a short-range communication protocol to communicate with the charging dock, such as Bluetooth, ZigBee, infrared transmission, and radio frequency (RF) communication. As such, if the infusion pump lacks longer range communication capabilities such as Wi-Fi, or cellular network technology, or if the user has not activated these communication capabilities, then the infusion pump may still communicate wirelessly with the charging dock. In some embodiments, the infusion pump may also use a short-range communication protocol to communicate wirelessly with nearby external devices such as a mobile device.

In some embodiments, the infusion pump may communicate wirelessly over longer distances to other external devices such as directly to a remote server.

In some embodiments, the charging dock may wirelessly charge the infusion pump. Data transfer may occur wirelessly from the infusion pump to the charging dock as well.

One illustrative example shown in FIG. 25 shows an infusion pump 600 communicating with different external devices. The infusion pump 600 may communicate, wirelessly and/or via a wired connection, with a mobile device 900 (e.g. a smartphone, tablet) having a display 910. The mobile device may be running an application that is designed for use with the infusion pump. The infusion pump 600 may communicate, wirelessly and/or via a wired connection, with a charging dock 800. The charging dock may have a charging connector 810 that is configured to physically connect with the infusion pump. The charging connector 810 may supply power to the battery of the infusion pump to charge the battery and, in some embodiments, may also allow for data transfer between the infusion pump and the charging dock. The charging dock itself may have the ability to communicate wirelessly and/or via wired connection with other external devices, such as a mobile device 900 or a remote server 1000. In some embodiments, other types of drug delivery devices may be used instead of an infusion pump.

According to one aspect, an infusion pump may directly and/or indirectly interact with a multitude of different parties that may utilize information from the infusion pump and/or send commands or other information to the infusion pump. Although the illustrative examples of FIG. 25 are shown with the portable embodiment of the infusion pump 600, it should be appreciated that other embodiments of the infusion pump may communicate with any of the parties or resources that are discussed below.

As a first example, information from an infusion pump may be sent directly or indirectly to a patient. A patient may obtain the information from a display 612 on the infusion pump, from a mobile device 900 that, e.g. may be running a companion application to the infusion pump, or from a remote server 1000. As an example, a patient may use a mobile device to obtain information from the remote server 1000, e.g. via an internet website or other program.

In some embodiments, the user may have access to a “patient service” feature 1200 that may serve as a type of customer service to the user. The user can connect with this service via, e.g. phone call, text, website, live chatting or other suitable communication format for assistance relating to the infusion pump and/or the medication. As an example, in some embodiments, the patient can use the patient service feature to receive training on how to use the infusion pump and/or any accessories relating to the infusion pump, how to troubleshoot any issues that may have arisen, or to ask any questions relating to the infusion pump or medication. In some embodiments, patients can use the patient service feature to help with issues relating to payment and/or insurance. The patient service may need to access information from the patient's infusion pump in order to help the patient with some of these issues. In some embodiments, the information may be obtained from the remote server 1000.

In some embodiments, an infusion pump may communicate directly or indirectly with a healthcare provider 1300, such as a hospital, clinic, and personnel such as a nurse or physician. The healthcare provider 1300 may obtain the information from a remote server 1000 or other external device such as a mobile device 900 that receives the information from the infusion pump 600. Or, the healthcare provider 1300 may be in direct communication with the infusion pump 600. Information that may be sent to a healthcare provider includes, but is not limited to, when a dosage was taken, how much medication was delivered, what the infusion delivery profile was, patient symptoms experienced, and so on. The healthcare provider may use the information to monitor patient adherence and/or to determine efficacy of the medication and/or the dosage regimen of the medication for the patient. From the information, the healthcare provider may, for example, choose to provide education and/or encouragement to the patient, and/or may adjust the patient's treatment. Communication between the infusion pump and the healthcare provider may be one-way or two-way communication. For example, in some embodiments, a healthcare provider may be able to send messages such as reminders or alerts to a patient via the infusion pump itself or to a mobile device that the patient uses in conjunction with the infusion pump, e.g. via an application running on the mobile device that may be specific for use with the infusion pump and/or the specific treatment that the infusion pump is being used for. Through the application on the mobile device, or through the infusion pump itself, a patient may be able to send questions or concerns directly to a healthcare provider, who may then provide replies back to the patient.

In some embodiments, the information communicated from the infusion pump may be integrated with a patient's Electronic Health Record 1600. The record may include information such as when a dosage was taken, how much medication was delivered, what the infusion delivery profile was, patient symptoms experienced, and so on.

In some embodiments, an infusion pump may communicate directly or indirectly with payers, also known as insurance companies. Payers may use information from the infusion pump to monitor aspects such as patient adherence, medication efficacy, and treatment regimen efficacy. In some embodiments, a payer may try to encourage or reward certain behaviors. For example, a payer may reward patients with good adherence by lowering rates or providing discounts. A payer may also encourage adherence by sending treatment reminders or alerts to patients and/or healthcare providers.

In some embodiments, information relayed through communication from and/or to an infusion pump may be used for data analytics, which may be used by a variety of parties. For example a supplier (e.g. a producer of a medication and/or an infusion pump) may use information from an infusion pump to determine what features are used most by users, and when (e.g. what buttons/control options are users selecting, on the infusion pump and/or on a companion app on a mobile device), what errors or issues are occurring, and so on. The information may be filterable into different categories such as age, gender, income, experience level, etc.

In some embodiments, information gathered for data analytics may be anonymous and free of PHI (patient health information). In other embodiments, however, the information may contain PHI.

In some embodiments, information gathered from an infusion pump may help provide performance of a medication. The inventors have appreciated that, outside of clinical trials, it can be difficult to assess the performance of a medication when it has been disseminated into the wider public. Communication from infusion pumps and other sources such as mobile devices and/or healthcare providers can help provide information regarding a medication and/or an infusion pump's performance. Information regarding a patient's symptoms and treatment progress may be gathered from patients, e.g. via an electronic symptom diary built into an infusion pump or into a companion app running on a mobile device, and/or may be gathered from a healthcare provider's notes taken during a patient's office visit. The gathered information may help to inform future formulations and/or infusion pump designs for the supplier, and positive performance may be used to help promote use of the medication.

In some embodiments, information relayed through communication from and/or to an infusion pump may be used to assist in supply chain management 1700. The information may include identification of what medication was used, and when (e.g. by sending a lot/batch number or other identifier associated with the medication). Information may also include the geographical region of medication use. Such information may help a medication supplier understand supply and demand of medication, e.g. in various regions of the world, as reflected by actual use of the medication (as compared to being limited to prescription fill information). This may help the supplier understand whether more or less medication should be stocked in certain regions, whether to increase marketing efforts in certain regions, and/or whether past marketing efforts have been effective at increasing demand.

According to one aspect, a mobile device may be used to control operation of a drug delivery device. In some embodiments, some or all of the user controls present on the drug delivery device may also be provided on the mobile device, e.g. via an application running on the mobile device. As such, between the drug delivery device and the mobile device running an application with user controls for controlling the drug delivery device, there may be some or complete redundancy of user controls. In some embodiments, the drug delivery device itself has little or no user controls at all, and most or all of the user controls are instead provided on the mobile device (e.g. via an application running on the mobile device).

In the illustrative embodiment shown in FIG. 25, the infusion pump 600 includes user controls 116′ for operating a drug delivery device. In some embodiments, the drug delivery device is an infusion pump 600, but it should be appreciated that other types of drug delivery devices may be used. The mobile device 900 may also run a companion app that provides a user with user controls on the display 910 of the mobile device 900 to permit the user to operate the infusion pump 600 using the mobile device. As such, in this embodiment, some or all of the user controls on the infusion pump are also available on the mobile device via an application.

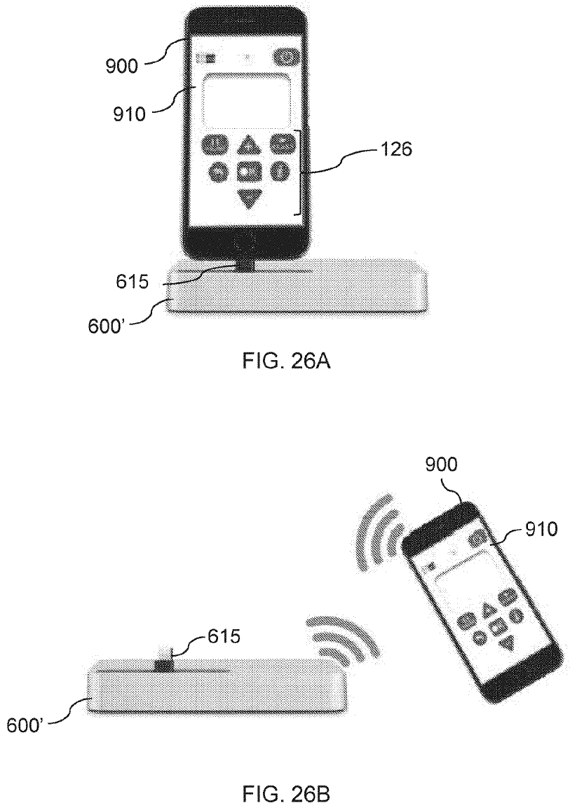

In the illustrative embodiment shown in FIG. 26A, a drug delivery device is provided with no user controls at all, or very few user controls. In some embodiments, the drug delivery device is an infusion pump 600′, but it should be appreciated that other types of drug delivery devices may be used. In the illustrative embodiment of FIG. 26A, a mobile device 900 may provide all or most of the user controls for operating the infusion pump 600′, e.g. via an application running on the mobile device that provides a display 910 having the user controls 126. In FIG. 26A, the mobile device 900 is shown docked to the infusion pump 600′ via a connector 615 such that the two are physically connected. Data communication may pass through the connector 615 such that a user may use the mobile device 900 to control the infusion pump 600′ while the mobile device 900 is docked to the infusion pump 600′. In some embodiments, the connector 615 may also transfer power, e.g. to charge the mobile device 900. As shown in FIG. 26B, in some embodiments, communication between the infusion pump 600′ and the mobile device 900 may also occur wirelessly.

The infusion pump 600′ may plug into a wall socket for power and/or may have a fully encapsulated battery that may be rechargeable. The infusion pump 600′ may be configured to sit on a table top during use, or may be configured to be worn by a user, e.g. clipped or otherwise coupled to an article of clothing.

In some embodiments, the motor coupling and pump coupling may be operatively connected when their respective axes of rotation are parallel and offset from one another. For example, the motor coupling and pump coupling may include spur, helical, or other suitable gears or transmission elements which are configured to contact and engage one another in a side-by-side arrangement. According to this embodiment, a pump engine and its rotor may be oriented parallel to an output shaft of a motor of a control unit when the control unit receives the pump engine. In this arrangement, the pump engine may be moved in a direction perpendicular to the output shaft to bring the pump engine into operative connection with a control unit. In some embodiments, the motor coupling and pump coupling may be operatively connected when their respective axes of rotation intersect with one another. That is, the motor coupling and pump coupling may be bevel gears or other suitable gears or transmission elements which are configured to transfer motion across angular intersections. For example, if the motor coupling and pump coupling include bevel gears, the axes of rotation may be perpendicular to one another. In such an arrangement, the pump engine may be received by a control unit in a direction perpendicular to an output shaft of a motor of the control unit. Of course, the pump engine and rotor may be oriented in any suitable position for reception by a control unit and operative connection of a motor coupling and a pump coupling, as the present disclosure is not so limited.

In some embodiments, a control unit may include a motor moving element configured to move the motor prior to the control unit receiving a pump engine. The motor moving element may move the motor linearly or rotationally so that a motor output shaft and motor coupling do not impede the reception of the pump engine. For example, a control unit with an openable compartment configured to receive a pump engine may have a multi-bar linkage connected to a lid covering the openable compartment that retracts the motor away from the compartment when the lid is opened. As another example, the motor may be disposed in a hinged compartment so that the motor may be rotated out of position until after the pump engine is received. Such an arrangement may ensure the pump engine is fully received and properly oriented in the control unit prior to operative connection of the motor to the pump engine. In some embodiments, the motor moving element may give an operator a mechanical advantage to align and operatively connect a motor coupling and a pump coupling.

FIGS. 27A and 27B depict front perspective views of another embodiment of a needle hub 1800 in a first position corresponding to a safety position and a second position corresponding to an infusion position, respectively. As shown in FIGS. 27A-27B, the needle hub includes a needle 1804 disposed in a needle housing 1802 between two wings 1810, 1820. Similarly to the embodiment of FIG. 18, the wings 1810, 1820 are configured to bend between multiple positions relative to the needle 1804. In particular, the wings are configured to bend away from one another and the needle 1804 from the safety position to an infusion position and an insertion position. In the infusion position, the wings may be perpendicular to the to an extension direction of the needle 1804 (for example, see FIG. 27B), such that the wings may be secured to a patient's skin with a medical adhesive or tape. In the insertion position, the wings may extend in a direction parallel to and opposite from the extension direction of the needle 1804, such that a user may grasp the wings to control the position of the needle to facilitate the insertion of the exposed needle into the patient's body.

According to the embodiment of FIGS. 27A-27B, each of the wings 1810, 1820 includes a first side 1812A, 1822A (corresponding to an exterior side relative to the needle 1804) and a second side 1812B, 1822B (corresponding to an interior side relative to the needle). A pair of ribs 1814 are disposed on the first side 1812A of the first wing 1810 and are configured to engage one or more corresponding ribs on the first side 1822A of the second wing 1820. Such an arrangement may be well suited to inhibit sliding of the wings relative to one another in a direction of extension of the needle 1804 when the first sides 1812A, 1822A are engaged with one another in the insertion position. As sliding is inhibited, roll of the needle 1804 may be inhibited as the needle is inserted into the patient's body. Similarly to the embodiment of FIG. 18, each of the wings also includes a flex area 1816, 1826 configured to increase the flexibility of the wings while moving between the safety position and the insertion position.

As shown in FIGS. 27A-27B, the needle hub 1800 also includes locking features 1818, 1828 disposed on the second sides 1812B, 1822B of each of the wings 1810, 1820. In particular, an annular receptacle 1818 is disposed on the second side 1812B of the first wing 1810, and an annular ring 1828 is disposed on the second side 1822B of the second wing 1820. The annular ring is configured to be received in the annular receptacle, where interference features on the annular receptacle 1818 and annular ring 1828 engage to lock the first wing to the second wing. Accordingly, when the second sides of the wings are pushed together, the wings may be locked together around the needle in a closed position. In the closed position, the needle may be inaccessible as the wings block and cover the needle 1804. In some cases, the locking features may be configured to effectively lock the wings 1810, 1820 together permanently. Accordingly, after an infusion process, the needle 1804 may be removed from a patient, and the wings may be folded to the closed position around the needle. When the wings are locked together in the closed position, the needle hub 1800 may be more easily handled and disposed of after an infusion process. Of course, in other embodiments, the wings may be releasable from one another when in the closed position, as the present disclosure is not so limited.

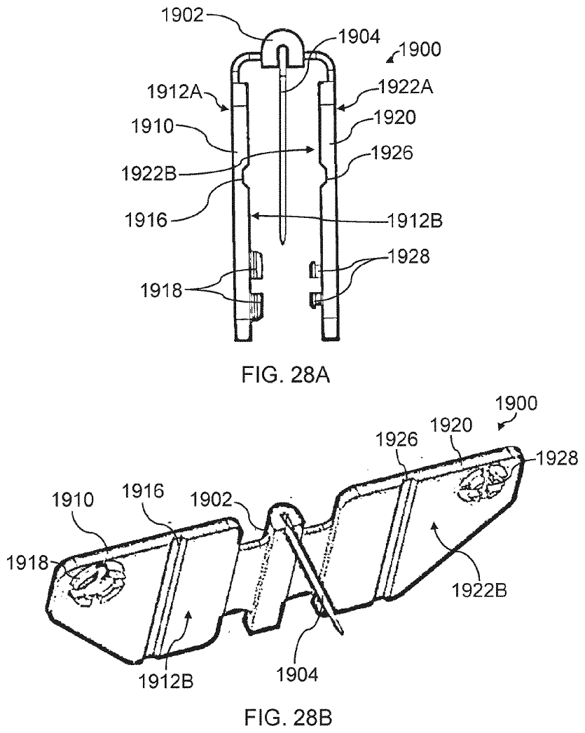

FIGS. 28A-28B depict a front view and a perspective view of another embodiment of a needle hub 1900 in a first position (e.g., safety position) and second position (e.g., infusion position), respectively. Similarly to the embodiment of FIGS. 27A-27B, the hub includes a needle 1904 disposed in a needle housing 1902 and two wings 1910, 1920 configured to move between safety, infusion, insertion, and closed positions. Each of the wings includes a first side 1912A, 1922A, and a second side 1912B, 1922B. A flex area 1916, 1926 on each wing facilitates the folding of the wings between the various positions.

According to the embodiment of FIGS. 28A-28B, the wings include locking features 1918, 1928 configured to lock the wings 1910, 1920 in a closed position. In particular, the second side 1912A of the first wing 1910 includes a female cavity 1918 having a plurality of snap ledges. The second side 1922B of the second wing 1920 includes a male post 1928 with a plurality of snap teeth. The snap teeth and snap ledges are configured to flex as the wings are moved into the closed position, where the snap teeth and snap ledges snap into position once the wings are fully in the closed position. Once in the closed position, the first wing and second wing may be locked together permanently or semi-permanently. The female cavity and male post may have any suitable number of snap ledges and snap teeth respectively. Additionally, the female cavity and male post may be disposed on opposite wings and on any suitable corresponding sides, as the present disclosure is not so limited in this regard (e.g., the female cavity may be disposed on the second wing and the male post may be disposed on the first wing).

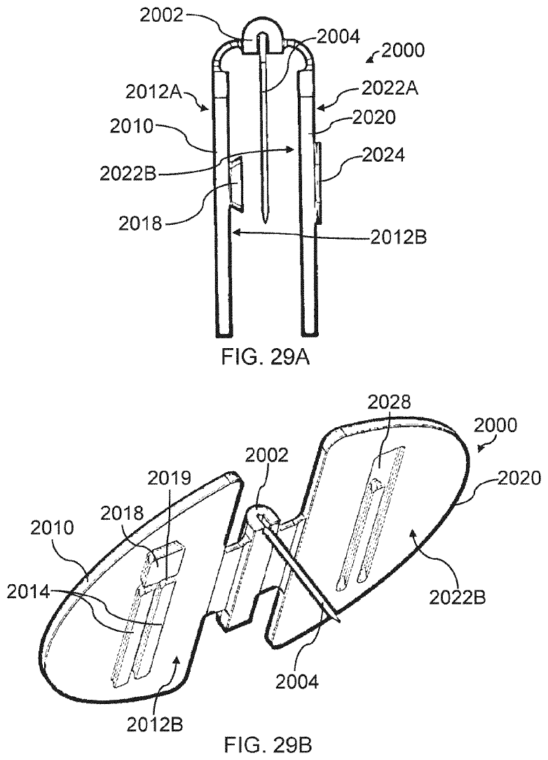

FIGS. 29A-29B depict a front view and a perspective view of another embodiment of a needle hub 2000 in a first position (e.g., safety position) and second position (e.g., infusion position), respectively. Similarly to previously discussed embodiments, the hub includes a needle 2004 disposed in a needle housing 2002 and two wings 2010, 2020 configured to move between safety, infusion, insertion, and closed positions. Each of the wings includes a first side 2012A, 2022A, and a second side 2012B, 2022B. In contrast to previously discussed embodiments, the wings 2010, 2020 are configured with a semi-circular shape instead of a triangular shape. Such an arrangement may increase the surface area of the wings available for grasping by a user,

According to the embodiment of FIGS. 29A-29B, the needle hub 2000 includes locking features 2014, 2018, 2028 which allow the wings to be secured in a closed position or an insertion position. As shown in FIG. 29B, the first wing 2010 includes a pair of elongated projections 2014 and a square shaped projection 2018 separated by a gap 2019 disposed on the second side 2012B of the first wing. A corresponding receptacle 2028 is disposed on the second side 2022B of the second wing 2020, which is configured to receive the projections 2014, 2018 on the first wing. As shown in FIG. 29A, the projections are flanged such that when the projections engage the receptacle 2028 the wings are retained together in the closed position. The gap 2019 is sized and shaped to receive the needle 2004, such that the needle does not interfere with the engagement of the projections 2014, 2018 and the receptacle 2028 as the wings are moved to the closed position. In the embodiment of FIGS. 29A-29B, the projections 2014, 2018 define a first wing receptacle in the first side 2012A of the first wing 2010. Likewise, the receptacle 2028 defines a second wing projection 2024 on the first side 2022A of the second wing 2020. Accordingly, when the wings are moved to an insertion position, the second wing projection 2024 and the first wing receptacle may engage one another to inhibit relative sliding of the first wing 2010 and the second wing 2020.

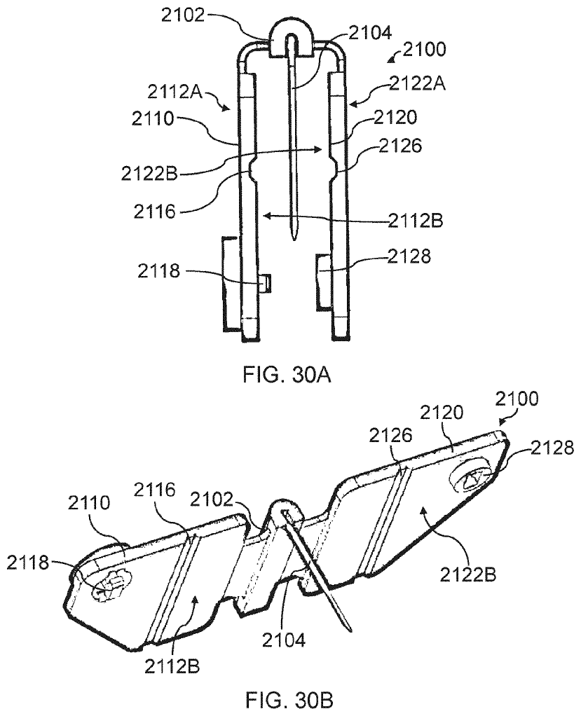

FIGS. 30A-30B depict a front view and a perspective view of another embodiment of a needle hub 2100 in a first position (e.g., safety position) and second position (e.g., infusion position), respectively. Similarly to the embodiments described previously, the hub includes a needle 2104 disposed in a needle housing 2102 and two wings 2110, 2120 configured to move between safety, infusion, insertion, and closed positions. Each of the wings includes a first side 2112A, 2122A, and a second side 2112B, 2122B. A flex area 2116, 2126 on each wing facilitates the folding of the wings between the various positions.

According to the embodiment of FIGS. 30A-30B, the first wing 2110 includes a receptacle 2114 on a first side 2112A and T-post 2118 on a second side 2112B. The second wing 2112 includes a T-post receptacle 2128 configured to receive the T-post 2118 when the wings are moved toward a closed position. When the wings are in the closed position, the wings may be locked together by the T-post and the T-post receptacle. According to the embodiment of FIGS. 30A-30B, the T-post 2118 and the T-post receptacle 2128 are positioned on the wings such that the needle 2104 does not interfere with the engagement of the T-post and T-post receptacle when the wings are in the closed position.

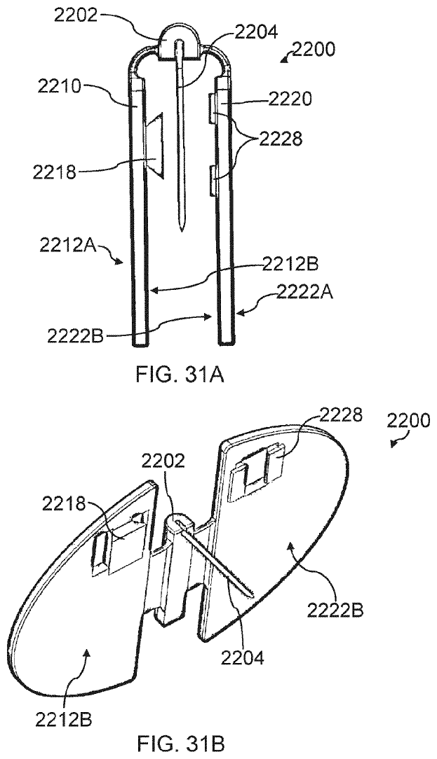

FIGS. 31A-31B depict a front view and a perspective view of another embodiment of a needle hub 2200 in a first position (e.g., safety position) and second position (e.g., infusion position), respectively. Similarly to the embodiments described previously, the hub includes a needle 2204 disposed in a needle housing 2202 and two wings 2210, 2220 configured to move between safety, infusion, insertion, and closed positions. Each of the wings includes a first side 2212A, 2222A, and a second side 2212B, 2222B. Similar to the embodiments of FIGS. 29A-29B, the wings 2210, 2220 of the needle hub are arranged with a semi-circular or semi-elliptical shape.

According to the embodiment of FIGS. 31A-31B, the first wing 2210 includes a wedge post 2218 disposed on the second side 2212B of the first wing. Correspondingly, the second wing 2220 includes a wedge post receptacle 2228 that includes two flexible ledges configured to selectively engage the wedge post 2218. In particular, as the wings 2210, 2220 are moved toward a closed position to enclose the needle 2204, the wedge post 2218 engages the wedge post receptacle 2228. As force is applied to the wings to move them toward the closed position, the two flexible ledges are moved outward relative to the wedge post receptacle to allow the wedge post to enter the wedge post receptacle. Once engaged, the wedge post and flexible ledges may lock the wings together permanently or semi-permanently. Of course, in some embodiments, the wedge post and wedge post receptacle may be releasable relative to one another once engaged, as the present disclosure is not so limited.

It should be noted that while particular embodiments of a needle hub are described with reference to FIGS. 27A-31B, the features of the wings such as the flex areas and locking features may be selectively interchanged or combined to provide a needle hub with characteristic desirable for a particular infusion application. For example, a flex area may be included on the semi-circular or semi-elliptical wings of the needle hub of FIGS. 31A-31B. As another example, the projections and receptacles of the needle hub of FIGS. 29A-29B may be included on the triangularly shaped wings of other embodiments. Thus, the features described with reference to FIGS. 27A-31B may be combined in any suitable combination, as the present disclosure is not so limited.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1-107. (canceled)

108. A system comprising:
a drug delivery device having a housing, a battery, and a controller;
a charging dock configured to cooperate with the drug delivery device to provide power to the battery of the drug delivery device;
wherein is data is configured to be transferred between the drug delivery device and the charging dock, and
wherein the drug delivery device is operable to dispense a medication without being connected to the charging dock.

109. The system of claim 108, wherein the drug delivery device comprises an infusion pump.

110. The system of claim 108, wherein the drug delivery device comprises a pen injector.

111. The system of claim 108, wherein the data is configured to be transferred from the drug delivery device to the charging dock.

112. The system of claim 108, wherein the data is configured to be transferred from the charging dock to the drug delivery device.

113. The system of claim 108, wherein the data is configured to be transferred from the drug delivery device to the charging dock and from the charging dock to the drug delivery device.

114. The system of claim 108, wherein the data is configured to be transferred wirelessly.

115. The system of claim 108, wherein the data is configured to be transferred over a wired connection.

116. The system of claim 108, wherein the charging dock comprises a connector that is removably connectable to the drug delivery device, wherein power and data is transferrable through the connector.

117. The system of claim 108, wherein the drug delivery device is configured to be worn on clothing of a user.
